(19) United States
(12) Patent Application Publication
(10) Pub. No.: US 2022/0287815 A1
(43) Pub. Date: Sep. 15, 2022

(54) FERTILITY PREDICTION IN ANIMALS

(71) Applicants: DAIRY AUSTRALIA LIMITED, Southbank (AU); AGRICULTURE VICTORIA SERVICES PTY LTD, Bundoora (AU); GEOFFREY GARDINER DAIRY FOUNDATION LIMITED, Melbourne (AU)

(72) Inventors: Jennie Elizabeth Pryce, Ivanhoe (AU); Phuong Ngoc Ho, Thomastown (AU)

(73) Assignees: DAIRY AUSTRALIA LIMITED, Southbank (AU); AGRICULTURE VICTORIA SERVICES PTY LTD, Bundoora (AU); GEOFFREY GARDINER DAIRY FOUNDATION LIMITED, Melbourne (AU)

(21) Appl. No.: 17/629,502

(22) PCT Filed: Jul. 24, 2020
(86) PCT No.: PCT/AU2020/050756

§ 371 Date: Jan. 24, 2022

(30) Foreign Application Priority Data
Jul. 25, 2019 (AU) 2019902639

(51) Int. Cl. A61D 17/00; A01J 5/007; G01N 33/68

(52) U.S. Cl. CPC A61D 17/002; A01J 5/007; G01N 33/689; G01N 2570/00

(57) ABSTRACT

The present invention is directed to methods for fertility prediction in animals, and in particular dairy cows. The methods allow detection of the likelihood of conception upon insemination of a cow based on the analysis of properties of milk of the cow, and in particular the mid-infrared (MIR) spectrum of the milk. Such methods also enable selection of cows for insemination and fertility classification of cows. Software and systems for carrying out the methods of the invention are also provided. The present invention also provides methods for deriving reference MIR spectra representative cows with good or poor likelihoods of conception upon insemination. These reference MIR spectra can be used for fertility prediction in cows to be tested.

This application claims priority from Australian provisional patent application number 2019902639 filed on 25 Jul. 2019, the content of which is to be taken as incorporated herein by this reference.

### FIELD OF THE INVENTION

The present invention relates generally to methods for fertility prediction in animals, and in particular dairy cows. The methods allow detection of the likelihood of conception upon insemination of a cow based on the analysis of properties of milk of the cow, and in particular the mid-infrared (MIR) spectrum of the milk. Such methods also enable selection of cows for insemination and fertility classification of cows.

### BACKGROUND OF THE INVENTION

In the dairy industry, reproductive efficiency is measured in terms of the ability of a cow to achieve pregnancy. A cow that is able to efficiently reproduce is a key driver of profit in dairy farming as it allows farmers to quickly breed cows after calving with a minimum number of inseminations per cow. Ultimately, the challenge is to achieve pregnancies in a timely and cost effective manner as both aspects affect profitability through influence on milk production, lifetime productivity of cows, herd expansion, culling rate, and availability of replacement stock.

Unfortunately, reproductive efficiency in cows has been greatly affected by declining fertility over the last few decades. Declining fertility is evidenced by decreased oestrus detection rates, conception rates, and an increased number of services per conception. Multiple factors have been reported to be associated with variation in conception rates. Non-genetic factors include quality and quantity of bull semen, age, body condition, energy balance, rumen undegradable protein, milk yield, health status of the cow, days post-calving, heat stress, lameness, and insemination season. Additive genetic effects have been predicted to account for about 2.3% of the phenotypic variation in conception rate.

For example, it has been established that declining fertility is particularly a challenge in high yielding cows due to genetic merit and nutritional management that are optimised towards lactation. That is, cows tend to prioritise nutrient mobilisation towards milk production over fertility in early lactation and this prioritisation of nutrients towards milk production also goes beyond the early lactation in high yielding cows. The prioritisation is genetically influenced thereby resulting in the body concentrating on milk production rather than the restoration of ovarian function and subsequent conception.

Despite the large efforts that have been made on investigating factors related to conception rate, comparatively few studies have attempted to predict the outcome of an individual insemination event (i.e., pregnant versus open). Prior knowledge of how likely a cow is to get pregnant, once inseminated, would enable farmers to optimize breeding decisions. For example, sexed or premium bull semen could be used for cows predicted with a high likelihood of conception, whereas cows with predicted poor fertility could be mated using semen from beef bulls, multiple doses, or with semen from bulls of known high genetic merit for fertility.

Accordingly, there is a need to develop methods for fertility prediction in dairy cows for improving farm management practices and optimising reproductive herd outcomes.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY OF THE INVENTION

The present invention arises out of studies conducted on dairy cows from commercial herds. The cows have been segregated into different groups based on their previous conception outcomes. Segregation in this manner has established that the mid-infrared (MIR) spectrum of their milk can provide a reference for predicting future conception outcomes for other cows. The segregation protocol has also enabled the identification of further properties of their milk, and properties of the cows per se, which, when combined MIR spectrum data, also provide a reference for predicting future conception outcomes for other cows. In effect, information relating to these properties in a cow's earlier lactation can forward predict future fertility and conception events in the cow.

Accordingly, in a first aspect the present invention provides a method of determining the likelihood of conception upon insemination of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination; and/or

comparing a mid-infrared (MIR) spectrum of milk of the cow with a second reference MIR spectrum, wherein the second reference MIR spectrum is representative of a cow or cows having a poor likelihood of conception upon insemination; and

determining the likelihood of conception upon insemination of the cow on the basis of the comparison,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and/or the second reference MIR spectrum are not derived from a cow or cows which have conceived following two or more inseminations and which did not conceive but had more than one insemination event at last mating season.

In some embodiments, the cow will have a good likelihood of conception upon insemination if the MIR spectrum of the milk of the cow is more consistent with the first reference MIR spectrum than with the second reference MIR spectrum. In some embodiments, the insemination is a second insemination.

In some embodiments, the cow will have a poor likelihood of conception upon insemination if the MIR spectrum of the milk of the cow is more consistent with the second reference MIR spectrum than with the first reference MIR spectrum. In some embodiments, the insemination is a first insemination.

In some embodiments, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

In some embodiments, the MIR spectrum of the milk of the cow is pre-treated prior to the comparison. In one embodiment, the pre-treatment is removal of spectral regions 2998 to 3998 cm⁻¹, 1615 to 1652 cm⁻¹, and 649 to 925 cm⁻¹.

In some embodiments, the method further comprises:

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination; and/or

comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and

determining the likelihood of conception upon insemination of the cow on the basis of the comparison,

wherein the first reference for the one or more further properties of the milk is derived from a cow or cows which have conceived at first insemination,

wherein the second reference for the one or more further properties of the milk is derived from a cow or cows which did not conceive within a previous mating season and had only one insemination event, and

wherein the first reference and/or the second reference for the one or more further properties of the milk are not derived from a cow or cows which have conceived following two or more inseminations and which did not conceive but had more than one insemination event at last mating season.

In some embodiments, the one or more further properties of the milk comprise somatic cell count (SCC), fat content, protein content, lactose content, and fatty acid content.

In some embodiments, the method further comprises:

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination; and/or

comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination; and

determining the likelihood of conception upon insemination of the cow on the basis of the comparison,

wherein the first reference for the one or more properties of the cow is derived from a cow or cows which have conceived at first insemination,

wherein the second reference for the one or more properties of the cow is derived from a cow or cows which did not conceive within a previous mating season and had only one insemination event, and

wherein the first reference and/or the second reference for the one or more properties of the cow are not derived from a cow or cows which have conceived following two or more inseminations and which did not conceive but had more than one insemination event at last mating season.

In some embodiments, the one or more properties of the cow comprise:

(i) milk yield (MY) on the day of obtaining the milk of the cow;

(ii) previous lactation (305-day) milk yield;

(iii) previous lactation (305-day) fat yield;

(iv) previous lactation (305-day) protein yield;

(v) days in milk (DIM) of the cow on the day of obtaining the milk of the cow;

(vi) days from calving to insemination (DAI) of the cow;

(vii) calving age of the cow from a previous insemination;

(viii) fertility genomic estimated breeding value (GEBV); and

(ix) genotype of the cow.

In some embodiments, the milk of the cow is obtained from the cow before intended insemination.

In a second aspect, the present invention provides a method of determining the likelihood of conception upon insemination of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination, and/or comparing a mid-infrared (MIR) spectrum of milk of the cow with a second reference MIR spectrum, wherein the second reference MIR spectrum is representative of a cow or cows having a poor likelihood of conception upon insemination; and

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination, and/or comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and/or

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination, and/or comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of each comparison,

wherein the first reference MIR spectrum, the first reference for the one or more further properties of the milk, and the first reference for the one or more properties of the cow, are derived from a cow or cows which have conceived at first insemination,

wherein the second reference MIR spectrum, the second reference for the one or more further properties of the milk, and the second reference for the one or more properties of the cow, are derived from a cow or cows which did not conceive within a previous mating season and had only one insemination event, and

wherein the first reference MIR spectrum, the first reference for the one or more further properties of the milk, the first reference for the one or more properties of the cow, the second reference MIR spectrum, the second reference for the one or more further properties of the milk, and the second reference for the one or more properties of the cow, are not derived from a cow or cows which have conceived following two or more inseminations and which did not conceive but had more than one insemination event at last mating season.

In some embodiments of the second aspect of the invention, the cow will have a good likelihood of conception upon insemination if the MIR spectrum of the milk of the cow is more consistent with the first reference MIR spectrum than with the second reference MIR spectrum. In some embodiments, the insemination is a second insemination.

In some embodiments of the second aspect of the invention, the cow will have a poor likelihood of conception upon insemination if the MIR spectrum of the milk of the cow is more consistent with the second reference MIR spectrum than with the first reference MIR spectrum. In some embodiments, the insemination is a first insemination.

In some embodiments of the second aspect of the invention, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

In some embodiments of the first and second aspects of the invention, the method further comprises selecting a cow for artificial insemination on the basis that it has a good likelihood of conception upon insemination.

In a third aspect, the present invention provides a method of selecting a dairy cow for artificial insemination, the method comprising:

comparing a mid-infrared (MIR) spectrum of milk of the cow with a first reference MIR spectrum, wherein the first reference MIR spectrum is representative of a cow or cows having a good likelihood of conception upon insemination; and/or

comparing a mid-infrared (MIR) spectrum of milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

selecting the cow for artificial insemination on the basis of the likelihood of conception,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In some embodiments of the third aspect of the invention, the cow will have a good likelihood of conception upon insemination if the MIR spectrum of the milk of the cow is more consistent with the first reference MIR spectrum than with the second reference MIR spectrum. In some embodiments, the insemination is a second insemination.

In some embodiments of the third aspect of the invention, the cow will have a poor likelihood of conception upon insemination if the MIR spectrum of the milk of the cow is more consistent with the second reference MIR spectrum than with the first reference MIR spectrum. In some embodiments, the insemination is a first insemination.

In some embodiments of the third aspect of the invention, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

In some embodiments of the third aspect of the invention, the method further comprises:

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination; and/or

comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and

determining the likelihood of conception upon insemination of the cow on the basis of the comparison; and

selecting the cow for artificial insemination on the basis of the likelihood of conception,

wherein the first reference for the one or more further properties of the milk is derived from a cow or cows which have conceived at first insemination,

wherein the second reference for the one or more further properties of the milk is derived from a cow or cows which did not conceive within a previous mating season and had only one insemination event, and

wherein the first reference and/or the second reference for the one or more further properties of the milk are not derived from a cow or cows which have conceived following two or more inseminations and which did not conceive but had more than one insemination event at last mating season.

In some embodiments of the third aspect of the invention, the method further comprises:

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination; and/or

comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of the comparison; and

selecting the cow for artificial insemination on the basis of the likelihood of conception,

wherein the first reference for the one or more properties of the cow is derived from a cow or cows which have conceived at first insemination,

wherein the second reference for the one or more properties of the cow is derived from a cow or cows which did not conceive within a previous mating season and had only one insemination event, and

wherein the first reference and/or the second reference for the one or more properties of the cow are not derived from a cow or cows which have conceived following two or more inseminations and which did not conceive but had more than one insemination event at last mating season.

In a fourth aspect, the present invention provides a method of selecting a dairy cow for artificial insemination, the method comprising:

comparing a mid-infrared (MIR) spectrum of milk of the cow with a first reference MIR spectrum, wherein the first reference MIR spectrum is representative of a cow or cows having a good likelihood of conception upon insemination, and/or comparing a mid-infrared (MIR) spectrum of milk of the cow with a second reference MIR spectrum, wherein the second reference MIR spectrum is representative of a cow or cows having a poor likelihood of conception upon insemination; and

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination, and/or comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and/or

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination, and/or comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of each comparison; and

selecting the cow for artificial insemination on the basis of the likelihood of conception,

wherein the first reference MIR spectrum, the first reference for the one or more further properties of the milk, and the first reference for the one or more properties of the cow, are derived from a cow or cows which have conceived at first insemination,

wherein the second reference MIR spectrum, the second reference for the one or more further properties of the milk, and the second reference for the one or more properties of the cow, are derived from a cow or cows which did not conceive within a previous mating season and had only one insemination event, and

wherein the first reference MIR spectrum, the first reference for the one or more further properties of the milk, the first reference for the one or more properties of the cow, the second reference MIR spectrum, the second reference for the one or more further properties of the milk, and the second reference for the one or more properties of the cow, are not derived from a cow or cows which have conceived following two or more inseminations and which did not conceive but had more than one insemination event at last mating season.

In some embodiments of the fourth aspect of the invention, the cow will have a good likelihood of conception upon insemination if the MIR spectrum of the milk of the cow is more consistent with the first reference MIR spectrum than with the second reference MIR spectrum. In some embodiments, the insemination is a second insemination.

In some embodiments of the fourth aspect of the invention, the cow will have a poor likelihood of conception upon insemination if the MIR spectrum of the milk of the cow is more consistent with the second reference MIR spectrum than with the first reference MIR spectrum. In some embodiments, the insemination is a first insemination.

In some embodiments of the fourth aspect of the invention, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

In a fifth aspect, the present invention provides a method of classifying the fertility of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination; and/or

comparing a mid-infrared (MIR) spectrum of milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In some embodiments of the fifth aspect of the invention, the cow will have a good likelihood of conception upon insemination if the MIR spectrum of the milk of the cow is more consistent with the first reference MIR spectrum than with the second reference MIR spectrum. In some embodiments, the insemination is a second insemination.

In some embodiments of the fifth aspect of the invention, the cow will have a poor likelihood of conception upon insemination if the MIR spectrum of the milk of the cow is more consistent with the second reference MIR spectrum than with the first reference MIR spectrum. In some embodiments, the insemination is a first insemination.

In some embodiments of the fifth aspect of the invention, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

In some embodiments of the fifth aspect of the invention, the method further comprises:

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination; and/or

comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination;

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference for the one or more further properties of the milk is derived from a cow or cows which have conceived at first insemination,

wherein the second reference for the one or more further properties of the milk is derived from a cow or cows which did not conceive within a previous mating season and had only one insemination event, and

wherein the first reference and the second reference for the one or more further properties of the milk are not derived from a cow or cows which have conceived following two or more inseminations and which did not conceive but had more than one insemination event at last mating season.

In some embodiments of the fifth aspect of the invention, the method further comprises:

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination; and/or

comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference for the one or more properties of the cow is derived from a cow or cows which have conceived at first insemination,

wherein the second reference for the one or more properties of the cow is derived from a cow or cows which did not conceive within a previous mating season and had only one insemination event, and

wherein the first reference and the second reference for the one or more properties of the cow are not derived from a cow or cows which have conceived following two or more inseminations and which did not conceive but had more than one insemination event at last mating season.

In a sixth aspect, the present invention provides a method of classifying the fertility of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination, and/or comparing a mid-infrared (MIR) spectrum of milk of the cow with a second reference MIR spectrum, wherein the second reference MIR spectrum is representative of a cow or cows having a poor likelihood of conception upon insemination; and

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination, and/or comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and/or

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination, and/or comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination

determining the likelihood of conception upon insemination of the cow on the basis of each comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference MIR spectrum, the first reference for the one or more further properties of the milk, and the first reference for the one or more properties of the cow, are derived from a cow or cows which have conceived at first insemination,

wherein the second reference MIR spectrum, the second reference for the one or more further properties of the milk, and the second reference for the one or more properties of the cow, are derived from a cow or cows which did not conceive within a previous mating season and had only one insemination event, and

wherein the first reference MIR spectrum, the first reference for the one or more further properties of the milk, the first reference for the one or more properties of the cow, the second reference MIR spectrum, the second reference for the one or more further properties of the milk, and the second reference for the one or more properties of the cow, are not derived from a cow or cows which have conceived following two or more inseminations and which did not conceive but had more than one insemination event at last mating season.

In some embodiments of the sixth aspect of the invention, the cow will have a good likelihood of conception upon insemination if the MIR spectrum of the milk of the cow is more consistent with the first reference MIR spectrum than with the second reference MIR spectrum. In some embodiments, the insemination is a second insemination.

In some embodiments of the sixth aspect of the invention, the cow will have a poor likelihood of conception upon insemination if the MIR spectrum of the milk of the cow is more consistent with the second reference MIR spectrum than with the first reference MIR spectrum. In some embodiments, the insemination is a first insemination.

In some embodiments of the sixth aspect of the invention, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

In a seventh aspect, the present invention provides software for use with a computer comprising a processor and memory for storing the software, the software comprising a series of coded instructions executable by the processor to carry out the method of any one of the first to sixth aspects of the invention.

In an eighth aspect, the present invention provides a software distribution means comprising the software of the seventh aspect of the invention.

In a ninth aspect, the present invention provides a system for determining the likelihood of conception upon insemination of a dairy cow, for classifying the fertility of a dairy cow, or for selecting a dairy cow for artificial insemination, the system comprising:

a processor;

a memory; and

software resident in the memory accessible to the processor, the software comprising a series of coded instructions executable by the processor to carry out the method of any one of the first to sixth aspects of the invention.

In a tenth aspect, the present invention provides software for use with a computer comprising a processor and memory for storing the software, the software comprising a series of coded instructions for executing a computer process by the processor, wherein the computer process determines the likelihood of conception upon insemination of a dairy cow, and wherein the computer process comprises:

receiving, inputting or accessing a mid-infrared (MIR) spectrum of milk of the cow;

comparing the mid-infrared (MIR) spectrum of the milk of the cow with a first reference MIR spectrum, wherein the first reference MIR spectrum is representative of a cow or cows having a good likelihood of conception upon insemination; and/or

comparing the mid-infrared (MIR) spectrum of the milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In an eleventh aspect, the present invention provides software for use with a computer comprising a processor and memory for storing the software, the software comprising a series of coded instructions for executing a computer process by the processor, wherein the computer process selects a dairy cow for artificial insemination, and wherein the computer process comprises:

receiving, inputting or accessing a mid-infrared (MIR) spectrum of milk of the cow;

comparing the mid-infrared (MIR) spectrum of the milk of the cow with a first reference MIR spectrum, wherein the first reference MIR spectrum is representative of a cow or cows having a good likelihood of conception upon insemination; and/or

comparing the mid-infrared (MIR) spectrum of the milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

selecting the cow for artificial insemination on the basis of the likelihood of conception,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In a twelfth aspect, the present invention provides software for use with a computer comprising a processor and memory for storing the software, the software comprising a series of coded instructions for executing a computer process by the processor, wherein the computer process classifies the fertility of a dairy cow, and wherein the computer process comprises:

receiving, inputting or accessing a mid-infrared (MIR) spectrum of milk of the cow;

comparing the mid-infrared (MIR) spectrum of the milk of the cow with a first reference MIR spectrum, wherein the first reference MIR spectrum is representative of a cow or cows having a good likelihood of conception upon insemination; and/or

comparing the mid-infrared (MIR) spectrum of the milk of the cow with a second reference MIR spectrum, wherein the second reference MIR spectrum is representative of a cow or cows having a poor likelihood of conception upon insemination; and

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In a thirteenth aspect, the present invention provides a software distribution means comprising the software of any one of the tenth to twelfth aspects of the invention.

In a fourteenth aspect, the present invention provides a system for determining the likelihood of conception upon insemination of a dairy cow, the system comprising:

a processor;

a memory; and

software resident in the memory accessible to the processor, the software comprising a series of coded instructions for executing a computer process by the processor, wherein the computer process determines the likelihood of conception upon insemination of the dairy cow, and wherein the computer process comprises:

receiving, inputting or accessing a mid-infrared (MIR) spectrum of milk of the cow;

comparing the mid-infrared (MIR) spectrum of the milk of the cow with a first reference MIR spectrum, wherein the first reference MIR spectrum is representative of a cow or cows having a good likelihood of conception upon insemination; and/or

comparing the mid-infrared (MIR) spectrum of the milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In a fifteenth aspect, the present invention provides a system for selecting a cow for artificial insemination, the system comprising:

a processor;

a memory; and

software resident in the memory accessible to the processor, the software comprising a series of coded instructions for executing a computer process by the processor, wherein the computer process selects a dairy cow for artificial insemination, and wherein the computer process comprises:

receiving, inputting or accessing a mid-infrared (MIR) spectrum of milk of the cow;

comparing the mid-infrared (MIR) spectrum of the milk of the cow with a first reference MIR spectrum, wherein the first reference MIR spectrum is representative of a cow or cows having a good likelihood of conception upon insemination; and/or

comparing the mid-infrared (MIR) spectrum of the milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

selecting the cow for artificial insemination on the basis of the likelihood of conception,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In a sixteenth aspect, the present invention provides a system for classifying the fertility of a dairy cow, the system comprising:

a processor;

a memory; and

software resident in the memory accessible to the processor, the software comprising a series of coded instructions for executing a computer process by the processor, wherein the computer process classifies the fertility of the dairy cow, and wherein the computer process comprises:

receiving, inputting or accessing a mid-infrared (MIR) spectrum of milk of the cow;

comparing the mid-infrared (MIR) spectrum of the milk of the cow with a first reference MIR spectrum, wherein the first reference MIR spectrum is representative of a cow or cows having a good likelihood of conception upon insemination; and/or

comparing the mid-infrared (MIR) spectrum of the milk of the cow with a second reference MIR spectrum, wherein the second reference MIR spectrum is representative of a cow or cows having a poor likelihood of conception upon insemination; and

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and the second reference MIR spectrum are not derived from a cow or cows which have conceived following two or more inseminations and which did not conceive but had more than one insemination event at last mating season.

In a seventeenth aspect, the present invention provides a method of deriving a first reference and/or a second reference for a mid-infrared (MIR) spectrum of milk of a dairy cow, the method comprising:

dividing a cohort of dairy cows into three groups based on previous insemination outcomes, wherein the first group are cows which have conceived at first insemination, wherein the second group are cows which did not conceive within a previous mating season and had only one insemination event, and wherein the third group are cows which have conceived following two or more inseminations and which did not conceive but had more than one insemination event at last mating season;

obtaining or accessing a mid-infrared (MIR) spectrum of milk of each cow of the first group and/or the second group;

comparing the MIR spectrum of the milk of a cow in the first group with the MIR spectrum of the milk of each other cow in the first group to derive a first reference MIR spectrum; and/or

comparing the MIR spectrum of the milk of a cow in the second group with the MIR spectrum of the milk of each other cow in the second group to derive a second reference MIR spectrum,

wherein the first reference MIR spectrum is representative of cows having a good likelihood of conception or good fertility, and wherein the second reference MIR spectrum is representative of cows having a poor likelihood of conception or poor fertility.

In some embodiments of the seventeenth aspect of the invention, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

In some embodiments of the seventeenth aspect of the invention, the MIR spectrum of the milk of each cow is pre-treated prior to the comparison. In some embodiments, the pre-treatment is removal of spectral regions 2998 to 3998 cm⁻¹, 1615 to 1652 cm⁻¹, and 649 to 925 cm⁻¹. In some embodiments, the pre-treatment is removal of outlier MIR spectra based on Mahalanobis distance. In some embodiments, the pre-treatment is application of first order Savitztky-Golay derivative.

In some embodiments of the seventeenth aspect of the invention, the method further comprises:

obtaining or accessing one or more further properties of the milk of each cow of the first group and/or the second group, wherein the one or more further properties of the milk are related to fertility, and;

comparing the one or more further properties of the milk of a cow in the first group with the one or more further properties of the milk of each other cow in the first group to derive a first reference for the one or more further properties of the milk; and/or

comparing the one or more further properties of the milk a cow in the second group with the one or more further properties of the milk of each other cow in the second group to derive a second reference for the one or more further properties of the milk,

wherein the first reference for the one or more further properties of the milk is representative of cows having a good likelihood of conception or good fertility, and wherein the second reference for the one or more further properties of the milk is representative of cows having a poor likelihood of conception or poor fertility.

In some embodiments of the seventeenth aspect of the invention, the one or more further properties of the milk comprise somatic cell count (SCC), fat content, protein content, lactose content, and fatty acid content.

In some embodiments of the seventeenth aspect of the invention, the method further comprises:

obtaining or accessing one or more properties of each cow of the first group and/or the second group, wherein the one or more properties of each cow are related to fertility, and;

comparing the one or more properties of a cow in the first group with the one or more properties of each other cow in the first group to derive a first reference for the one or more properties of the cow; and/or

comparing the one or more properties of a cow in the second group with the one or more properties of each other cow in the second group to derive a second reference for the one or more properties of the cow,

wherein the first reference for the one or more properties of the cow is representative of cows having a good likelihood of conception or good fertility, and wherein the second reference for the one or more properties of the cow is representative of cows having a poor likelihood of conception or poor fertility

In some embodiments of the seventeenth aspect of the invention, the one or more properties of the cow comprise:

(i) milk yield (MY) on the day of obtaining the milk of the cow;

(ii) previous lactation (305-day) milk yield;

(iii) previous lactation (305-day) fat yield;

(iv) previous lactation (305-day) protein yield;

(v) days in milk (DIM) of the cow on the day of obtaining the milk of the cow;

(vi) days from calving to insemination (DAI) of the cow;

(vii) calving age of the cow from a previous insemination;

(viii) fertility genomic estimated breeding value (GEBV); and

(ix) genotype of the cow.

### BRIEF DESCRIPTION OF THE FIGURES

For a further understanding of the aspects and advantages of the present invention, reference should be made to the following detailed description, taken in conjunction with the accompanying figures which illustrate certain embodiments of the present invention.

FIG. 1—Plots showing a visual comparison of milk mid-infrared (MIR) spectra between “good”, “average” and “poor” fertility categorized groups of cows. A: “good” versus “poor” fertility cows. B: “average” versus “poor” fertility cows. C: “average” versus “good” fertility cows. The solid lines in each plot represent a typical pre-treated absorbance spectrum for a cow randomly taken from the dataset used in Example 1, while the circles are −log 10(p-values) associated with the F-statistic of the estimated differences between the different categories of fertility. The dashed lines in each plot represent the cut-off point for significance level. Left Y-axis: P-values obtained from pairwise comparison of MIR spectra of the different categories of fertility. Right Y-axis: Absorbance. X-axis: Range of wavenumbers.

FIG. 2—is a schematic diagram of a system according to an embodiment of the present invention.

FIG. 3—is a series of detailed schematic drawings of the components included in a processor according to various embodiments of the present invention. FIG. 3A shows a processor for determining the likelihood of conception upon insemination of a dairy cow, FIG. 3B shows a processor for selecting a dairy cow for artificial insemination, and FIG. 3C shows a processor for classifying the fertility of dairy cow.

FIG. 4—is a flow diagram of a method for determining the likelihood of conception upon insemination of a dairy cow according to an embodiment of the invention.

FIG. 5—is a flow diagram of a method for selecting a dairy cow for artificial insemination according to an embodiment of the invention.

FIG. 6—is a flow diagram of a method for classifying the fertility of dairy cow according to an embodiment of the invention.

FIG. 7—a graph showing the conception rate at first insemination (x-axis) of the herds used in the study in Example 1. The number of herds for each conception rate is shown on the y-axis.

FIG. 8—a graph showing the average conception rate to first insemination (x-axis) across the 39 herd-years (32 herds) used in the study in Example 2. The number of herd-years for each conception rate is shown on the y-axis.

FIG. 9—plots showing the correlation between observed herd-year mean conception rate to first insemination in the study in Example 2 and prediction accuracy of the models for identifying cows in that herd-year with good likelihood of conception to second insemination (A) and poor likelihood of conception to first insemination (B).

### DETAILED DESCRIPTION OF THE INVENTION

As set out above, the present invention is predicated, in part, on the identification of properties of milk of a dairy cow (and in particular the mid-infrared (MIR) spectrum of the milk), and properties of the cow from which the milk is derived, which serve as predictors of fertility and conception outcomes in the cow. The relevance of the properties as predictors has been identified through a unique segregation protocol of a cohort of dairy cows from commercial herds.

Accordingly, certain disclosed embodiments provide methods and systems that have one or more advantages. For example, some of the advantages of some embodiments disclosed herein include one or more of the following: improved methods for fertility prediction in dairy cows; improved methods for determining the likelihood of conception upon insemination of a dairy cow; improved methods for selecting dairy cows for insemination; improved methods for classifying the fertility of a dairy cow; methods which enhance farm management practices; methods which optimise reproductive herd management; methods for deriving reference values for one or more properties of a cow and milk obtained from the cow which are representative of cows having good or poor fertility; novel herd segregation methods enabling derivation of reference values for one or more properties of a cow and milk obtained from the cow which are representative of cows having good or poor fertility; and software and related systems for performing such methods; or the provision of a commercial alternative to existing methods and systems. Other advantages of some embodiments of the present disclosure are provided herein.

The unique herd segregation protocol adopted herein has enabled cows to be classified according to their predicted fertility status. While segregation of cows has been attempted in the past for such purposes, prediction accuracy has been much lower than that achieved by the present invention. The improved accuracy obtained by the present inventors is predicated in part on the segregation of cows for data analysis into extreme groups and excluding data obtained from cows which fall between these two extremes. Specifically, segregation was made based on previously observed conception events in a cohort of cows. The principle behind the segregation protocol is to group cows within the cohort on the basis of good (high) fertility or poor (low) fertility. The fertility classification can be made in any way provided it is reflective of the previously observed conception events of each cow in the cohort. The intent of this approach is to create a divergence of observations for various properties of milk of the cows, and optionally properties of the cows themselves, in order to train a prediction model for cow fertility.

For example, a segregation protocol according to an embodiment of the present invention groups cows in a cohort as follows: cows having been able to conceive at first insemination (extreme group 1—classified as having “good” fertility); those which had not conceived within a previous mating season and had only one insemination event (extreme group 2—classified as having “poor” fertility); and those which had conceived following two or more inseminations and which did not conceive (but had more than one insemination event) at last mating season (group 3—classified as having “average” fertility). The exclusion of data with respect to cows in group 3 has been instrumental in improving the ability to predict fertility, and determine conception likelihood, in cows.

Indeed, the concept of segregating cows from a cohort into good and poor fertility status prior to data analysis has enabled the identification of a reference with respect to one or more properties of milk obtained from cows, and one or more properties of the cows, which distinguish cows with predicted good likelihood of conception from those with predicted poor likelihood of conception. In particular, the mid-infrared (MIR) spectrum of the milk has been found to serve as a predictor of fertility and conception outcomes following insemination. In effect, comparing the MIR spectrum of a cow's earlier lactation with a reference MIR spectrum obtained from the segregation protocol can forward predict future fertility and conception events in the cow.

As used herein, the terms “fertility” and “conception” are interchangeable and generally mean the ability of a cow to become pregnant and produce offspring upon insemination. A cow having good fertility will have a good likelihood of conception upon insemination, and vice-versa. Alternatively, a cow having poor fertility will have a poor likelihood of conception upon insemination, and vice-versa.

The likelihood of conception upon insemination of a particular cow (i.e. a test cow) can be determined based on a comparison between the MIR spectrum of milk obtained from the cow, and optionally a comparison between one or more further properties of the milk and/or one or more properties of the cow from which the milk was obtained, with a reference for each property which has been predetermined, and has been derived, through use of a segregation protocol described herein.

The reference for a property, including a reference MIR spectrum, can be derived from an individual reference cow or from a cohort of cows. For example, a first reference for each property can be obtained from a cow known to have consistent good fertility each mating season. In one embodiment, such a cow would have previously conceived at first insemination. Similarly, a second reference for each property can be obtained from a cow known to have consistent poor fertility each mating season. In one embodiment, such a cow would be one which did not conceive within a previous mating season having had only one insemination event.

When the (predetermined) reference for a property, including a reference MIR spectrum, is derived from more than one cow, for example from a cohort of cows from a number of herds, an average for each property across the cohort may be obtained. For example, with respect to a MIR spectrum representing a cohort of cows having good fertility, each wavenumber in each spectrum of the representative cohort of good fertility cows is an average of that specific wavenumber across all cows in that fertility category.

A first reference for each property, which represents an average or consensus for each property, can be obtained from a cohort of cows known to have consistent good fertility each mating season. In one embodiment, each cow in such a cohort would have previously conceived at first insemination. Similarly, a second reference for each property, which represents an average or consensus for each property, can be obtained from a cohort of cows known to have consistent poor fertility each mating season. In one embodiment, each cow in such a cohort would be one which did not conceive within a previous mating season having had only one insemination event.

In some embodiments, when using a cohort of cows for deriving the first and second reference for each property (including the first reference MIR spectrum and second reference MIR spectrum), the cows may be from herds of the same breed, from herds which differ in breed, differ in physical location, or are crossbred.

The first reference and second reference for each property can be used to compare with the equivalent property of a cow for which the likelihood of conception is being determined (i.e. a test cow). In some embodiments, an MIR spectrum of the test cow, and optionally one or more further properties of the milk of the cow and/or a property of the cow itself, which is consistent with the first reference or second reference for each property will be indicative of a good likelihood or poor likelihood of conception upon insemination, respectively, in the cow being tested.

When deriving a reference for a property of milk of a cow, or a reference for a property of the cow itself, one may rely on historical data already collected for a cow or cohort of cows. Typically the historical data is stored in a database which can be interrogated. In this regard, only data with respect to conception information from previous lactations from cows which fall into the two extreme fertility groups (“good” fertility or “poor” fertility) is interrogated. If such historical data is not available then it must first be obtained from a cow or cohort of cows prior to interrogation and derivation of the reference.

Based on the segregation protocol adopted herein, it has been shown that the mid-infrared (MIR) spectrum of milk of a cow can predict fertility outcomes in the cow upon an insemination event. Accordingly, in a first aspect the present invention provides a method of determining the likelihood of conception upon insemination of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination; and/or

comparing a mid-infrared (MIR) spectrum of milk of the cow with a second reference MIR spectrum, wherein the second reference MIR spectrum is representative of a cow or cows having a poor likelihood of conception upon insemination; and

determining the likelihood of conception upon insemination of the cow on the basis of the comparison,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and/or the second reference MIR spectrum are not derived from a cow or cows which have conceived following two or more inseminations and which did not conceive but had more than one insemination event at last mating season.

A mid-infrared (MIR) spectrum of milk is obtained from infrared spectroscopy of the milk at defined wavelengths. For example, a recorded MIR spectrum will include numerous data points, with each point representing the absorption of infrared light through the milk at particular wavenumbers in the 400 to 4,000 cm⁻¹ region (2,500 to 25,000 nm). The complete infrared spectrum of the milk may first be obtained with only data from the mid-infrared range subsequently used for the analysis, or the MIR spectrum in the 400 to 4,000 cm⁻¹ region only of the milk may be obtained.

As would be understood by a person skilled in the art, infrared spectroscopy involves the interaction of infrared radiation with matter in the milk, and therefore exploits the differences in milk constitution that exists between different milk samples. Infrared spectroscopy of the milk may be performed using a standard benchtop infrared spectrophotometer available from commercial suppliers such as Bentley Instruments (Chaska, Minn., USA), Delta Instruments (Drachten, The Netherlands), Bruker Optics (Billerica, Minn., USA), JASCO (Eastland, Md., USA), Foss Analytics (Hillerød, Denmark), Agilent Technologies (Santa Clara, Calif., USA), and ABB Analytical (Zurich, Switzerland). The infrared spectrophotometer may also be a portable or handheld device such as those also available from the above suppliers. Such portable devices are useful for on-farm analysis of milk samples. Other sources of spectroscopy apparatus would be known to those skilled in the art.

The infrared spectrum of milk is recorded by passing a beam of infrared light through the milk. When the frequency of the IR is the same as the vibrational frequency of a bond or collection of bonds, absorption occurs. Examination of the transmitted light reveals how much energy was absorbed at each frequency (or wavelength), which can be used to quantify the abundance of molecules present in the milk. This measurement can be achieved by scanning the relevant wavelength range using a monochromator. Alternatively, the entire wavelength range is measured using a Fourier transform instrument and then a transmittance or absorbance spectrum is generated using a dedicated procedure.

In some embodiments, raw spectra of milk obtained over the 400 to 4,000 cm⁻¹ region may be subject to a pre-treatment before chemometric analysis. A pre-treatment is performed to eliminate regions of the spectra characterized by low signal to noise ratio resulting from high water absorption. In some embodiments, such spectral regions include 2998 to 3998 cm⁻¹, 1615 to 1652 cm⁻¹, and 649 to 925 cm⁻¹.

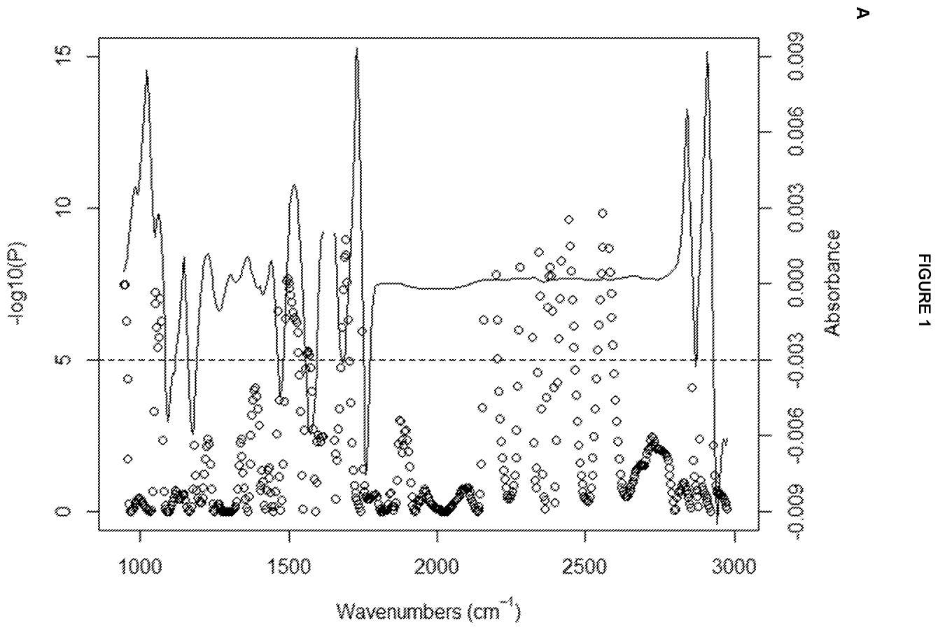

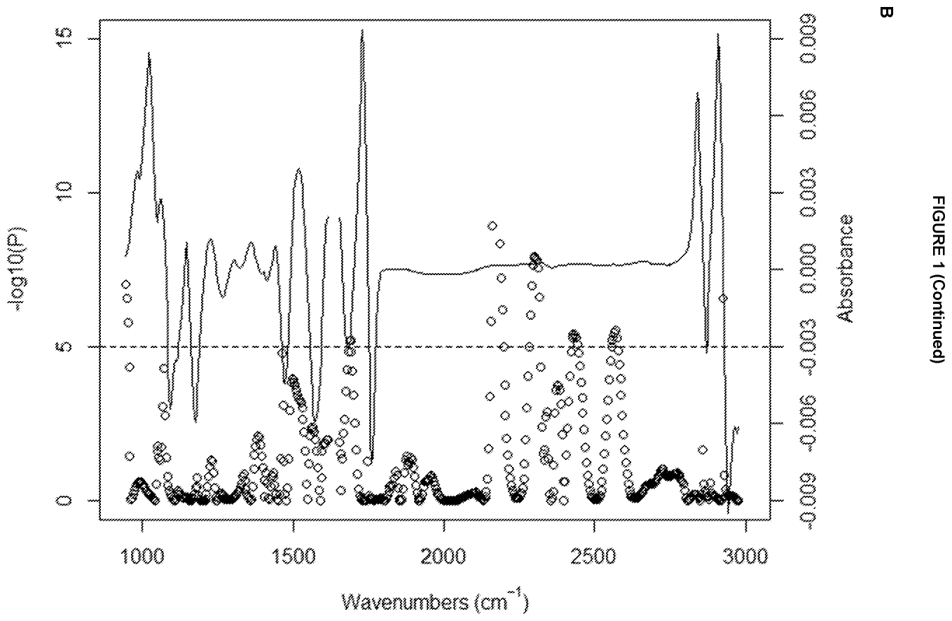

A first reference MIR spectrum or second reference MIR spectrum may be derived from milk obtained from an individual cow for which good or poor fertility has been assigned based on their previous conception record, as described above. Alternatively, MIR spectra derived from milk obtained from each cow in a cohort of cows for which good or poor fertility has been assigned based on their previous conception record, may be used to generate a consensus MIR spectra for the cohort. In effect, a first reference MIR spectrum will be representative of a cow or cows having consistent good fertility each mating season. In contrast, a second reference MIR spectrum will be representative of a cow or cows having consistent poor fertility each mating season. Representative MIR spectra are represented visually in FIG. 1.

For example, FIG. 1A is a MIR spectrum showing differences from an analysis of variance comparing the MIR spectra of “good fertility” cows and “poor fertility” cows. The circles in the spectrum are −log 10(p-values) associated with the F-statistic of the estimated difference between “good” and “poor” fertility cows. The F-statistic (or analysis of variance) has been used in this instance to provide a visual representation of the variance between the MIR spectra of “good fertility” cows and “poor fertility” cows. As can be seen from FIG. 1A, a significant amount of variation in predictive power of wavenumbers of the spectrum is observed. The line across the spectrum in FIG. 1A represents a typical absorbance spectrum pattern for a cow with likely differences between the two fertility categories highlighted by the individual circles across the spectrum.

Therefore, when determining the likelihood of conception upon insemination of a test cow, a MIR spectrum of milk of the test cow is obtained and is compared to the representative first reference MIR spectrum and/or second reference MIR spectrum. In some embodiments of the aspects of the invention, when the MIR spectrum of milk of the cow being tested is more consistent with the representative first reference MIR spectrum than with the second reference MIR spectrum, then the cow will have a good likelihood of conception. For example, the inventors have shown that consistency between the MIR spectrum of the milk of the cow being tested and the first reference MIR spectrum is a predictor of a good likelihood of conception upon second insemination of the cow being tested.

In some embodiments of the aspects of the invention, when the MIR spectrum of milk of the cow being tested is more consistent with the representative second reference MIR spectrum than with the first reference MIR spectrum, then the cow will have a poor likelihood of conception. For example, the inventors have shown that consistency between the MIR spectrum of the milk of the cow being tested and the second reference MIR spectrum is a predictor of a poor likelihood of conception upon first insemination of the cow being tested.

By “more consistent” is taken to mean the MIR spectrum of milk of the cow being tested has features (for example individual waveforms) which are similar to, or the same as, those of the first reference MIR spectrum or second reference MIR spectrum. Represented visually (through F-statistic analysis), when the MIR spectrum of the milk of the test cow is compared to a reference spectrum for a good fertility cow (i.e. a first reference MIR spectrum), if variance similar to that shown in FIG. 1A is observed (represented by the number of circles above the significance cut-off line) then it would suggest that the test cow has poor fertility. However, if the two spectra display minimal or no variance (i.e. the two spectra are more consistent with each other) across the wavenumbers then it would suggest that the test cow has good fertility. Similarly, when the MIR spectrum of milk of the test cow is compared to a reference spectrum for a poor fertility cow (i.e. a second reference MIR spectrum), if variance similar to that shown in FIG. 1A is observed then it would suggest that the test cow has good fertility. However, if the two spectra are consistent across the wavenumbers then it would suggest that the test cow has poor fertility.

In FIGS. 1B and 1C, the difference in MIR spectra between “average” and “poor” fertility cows, or “average” and “good” fertility cows, respectively, is shown. The lower level of variance observed in the MIR spectra between these categories of cows highlights the extreme variance which is observed between the “good” and “poor” fertility MIR spectra (FIG. 1A). This emphasises the value of the herd segregation protocol described above in providing meaningful reference MIR spectra for forward fertility prediction in cows.

As indicated above, the likelihood of conception upon insemination of the cow is determined based on a comparison between MIR spectra. In some embodiments of the aspects of the present invention, the likelihood determination may be obtained through a statistical comparison of the MIR spectra. Such a statistical comparison can be implemented through the use of any one of a number of algorithms which have, for example, the ability to compare MIR spectral features of each MIR spectrum being compared. In some embodiments of the aspects of the present invention, the MIR spectral features are individual waveforms of each MIR spectrum.

The algorithms automatically determine which features (or waveforms) of the MIR spectra best describe the likelihood of conception success. Representative algorithms include partial least squares regression (including partial least squares discriminant analysis (PLS-DA)), C4.5 decision trees, naive Bayes, Bayesian network, logistic regression, support vector machine, random forest, and rotation forest. These have been described in Hempstalk K et al., 2015, J. Dairy Sci., 98: 5262-5273. The invention is not limited by the aforementioned statistical algorithms.

Partial least squares regression (PLS; Geladi P and Kowalski B R, 1986, Anal. Chim. Acta, 185: 1-17) can be performed as a preprocessing step before training a machine learning algorithm; it works like principal component analysis (PCA) in that it transforms the data set into a new projection that represents the entire data set, and then chooses the C most informative axes (or “components”) in the new projection as features in the transformed data set. Where the PCA and PLS algorithms differ is that PLS takes into consideration the dependent variable when constructing its projection, but PCA does not. One advantage of using the dependent variable during learning is that the algorithm is able to perform regression using the projections it has calculated. A binary prediction (i.e., conceived or not) can be made by creating a regression model that predicts the probability (of conception) and returning true if the probability reaches a set threshold, or false otherwise. PLS-DA is a variant of partial least squares regression when the response variable is categorical, which is used to find the relationship between two matrices. It is one of the most well-known classification methods in chemometrics, metabolomics, and proteomics with an ability to analyze highly collinear data which is often a problem with conventional regression methods, for example, logistic regression (Gromski P S et al., 2015, Analytica Chimica Acta., 879: 10-23).

The C4.5 decision tree (Quinlan R, 1993, Programs for Machine Learning. Morgan Kaufmann Publishers, San Mateo, Calif., USA) builds a tree by evaluating the information gain of each feature (i.e., independent variable) and then creates a split (or decision) by choosing the most informative feature and dividing the records into left and right nodes of the tree. This process repeats until all of the records at a node belong to a single class (i.e., conceived or not) or the number of records reaches the threshold defined in the algorithm (i.e., a minimum of 2 instances per leaf). A prediction is made by traversing the tree using the values from the current instance and returning the majority class at the leaf node reached by the traversal. The tree prevents over-fitting by performing pruning to remove nodes that may cause error in the final model.

The naive Bayes algorithm “naively” assumes each feature is independent and builds a model based on Bayes' rule. It multiplies the probabilities of each feature belonging to each class (i.e., conceived or not) to generate a prediction. The probability for each feature is calculated by supplying the mean and standard deviation to a Gaussian probability density function, which are then multiplied together using Bayes' rule.

A Bayesian network classifier represents each feature as a node on a directed acyclic graph, each node containing the conditional probability distribution that can be used for class prediction. A Bayesian network assumes that each node is conditionally independent of its nondescendants, given its immediate parents. During calibration, the network structure is built by searching through the space of all possible edges and computing the log-likelihood of each resulting network as a measure of quality.

Linear regression is a common statistical technique used to express a class variable as a linear combination of the features. However, it is designed to predict a real numeric value and cannot handle a categorical or binary class (i.e., conceived or not). To overcome this, a model can be built for each class value that ideally predicts 1 for that class value, and 0 otherwise, and at prediction time assigns the class value whose model predicts the greatest probability. Unfortunately, regression functions are not guaranteed to produce a probability between 0 and 1, and so the target class must first be transformed into a new space before it is learned. This is achieved using a log-transform, and this regression method is known as logistic regression (Witten I H et al., 2011, Data Mining: Practical Machine Learning Tools and Techniques. Morgan Kaufmann, USA). In logistic regression, the weights are chosen to maximize the log likelihood (instead of reducing the squared error), by iteratively solving a sequence of weighted least-squares regression problems until the log-likelihood converges on the maximum. One algorithm in WEKA Machine Learning Workbench that performs this type of logistic regression is SimpleLogisticRegression, which by default uses boosting (M=500) to find the maximum log-likelihood, and cross-validation with greedy stopping (H=50) to ensure the algorithm stops boosting if no gains have been made in the last H iterations.

Support vector machines (SVM) can produce nonlinear boundaries (between classes) by constructing a linear boundary in a large, transformed version of the feature space (Hastie T et al., 2009, The Elements of Statistical Learning: Data Mining, Inference, and Prediction. Springer, New York, N.Y.). In practice, a soft margin boundary (Cortes C and Vapnik P, 1995, Mach. Learn., 20: 273-297) is used to prevent over-fitting; however, a hard margin is easier to visualize when describing SVM. In the hard margin case, the algorithm assumes that classes in the transformed space are linearly separable, and it is possible to generate a hyperplane that completely separates them. By employing a technique known as the kernel trick (Aizerman M A et al., 1964, Autom. Remote Control, 25: 821-837), SVM are able to generate nonlinear decision boundaries. This is possible because the kernel trick reduces the computational effort by estimating similarities of the transformed instances as a function of their similarities in the original space. One example of an SVM is SMO, sequential minimal optimization (Platt J, 1998, Pages 185-208 in Advances in Kernel Methods: Support Vector Learning. B. Scholkopf, C. J. Burges, and A. J. Smola, ed. MIT Press, Cambridge, Mass.), from WEKA (Witten I H et al., 2011, supra), which uses the sequential minimal optimization algorithm to increase the speed of finding the maximum-margin hyperplane.

Random forest (Breiman L, 2001, Mach. Learn., 45: 5-32) is an ensemble learner that creates a “forest” of decision trees, and predicts the most popular class estimated by the set of trees. Each tree is provided with a random set of training instances sampled with replacement from the entire training set. The intention of this step is to create a diverse set of trees. The algorithm differs from bagged decision trees (which also provide randomly selected subsets to each tree) because during training the algorithm randomly selects a subset of features available for selection at each split in the tree. One implementation of this algorithm is RandomForest in WEKA, which by default has an unlimited tree depth (maxDepth=0) and the number of features randomly selected into each subset=log 2(total number of features)+1. By default, this algorithm creates a forest of 10 trees (numTrees=10); however, this can be increased to 1,000 (numTrees=1000) to cater for poor accuracy when considering only 10 trees. The effect of increasing this parameter is that accuracy is improved, but also that the algorithm takes much longer to run.

Rotation forest (Rodriguez J J et al., 2006, IEEE Trans. Pattern Anal. Mach. Intell., 28: 1619-1630) is an ensemble learner similar to random forest except that PCA is applied to select the features for each tree (instead of random selection), and the components are all kept when the base classifier is trained. The classifier sees a “rotated” set of features in each tree in its forest. The intention is to create individual accuracy in the tree and diversity in the ensemble, compared with random forest, which aims only to create diversity in the ensemble. Results for a rotation forest learner have been shown to be as good as those of other ensemble learning schemes such as bagging, boosting, and random forests (Rodriguez J J et al., 2006, supra).

As indicated above, analysis of the MIR spectrum of the milk of a cow may also be combined with an analysis of one or more further properties of the milk of the cow as a predictor of fertility and conception outcomes. Accordingly, in some embodiments, the method of the first aspect of the present invention further comprises:

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination; and/or

comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and

determining the likelihood of conception upon insemination of the cow on the basis of the comparison,

wherein the first reference for the one or more further properties of the milk is derived from a cow or cows which have conceived at first insemination,

wherein the second reference for the one or more further properties of the milk is derived from a cow or cows which did not conceive within a previous mating season and had only one insemination event, and

wherein the first reference and/or the second reference for the one or more further properties of the milk are not derived from a cow or cows which have conceived following two or more inseminations and which did not conceive but had more than one insemination event at last mating season.

Determining a first reference or second reference for the one or more further properties of the milk has been described above.

In some embodiments, the one or more further properties of the milk obtained from the cow comprise somatic cell count (SCC), fat content, protein content, lactose content, and fatty acid content of the milk. Other properties of the milk are contemplated provided that they are related to, and contribute to, fertility outcomes in cows. Typically, these properties of the milk, including the MIR spectrum of the milk, are measured in a milk sample obtained from the cow. The properties can be measured on-farm or on-site provided the facility has the necessary resources to do so. Otherwise, the milk sample can be sent off-site for testing, for example at a suitably qualified laboratory testing facility. Indeed, a number of these milk properties must be routinely tested as a condition of milk sale.

The somatic cell count (SCC) of milk is a measure of the total number of cells per milliliter of a milk sample. Primarily, SCC is composed of leukocytes, or white blood cells, that are produced by the cow's immune system to fight an inflammation in the mammary gland, or mastitis. Therefore, SCC is an indicator of the quality of milk give that the number of somatic cells increases in response to pathogenic bacteria such as Staphylococcus aureus, which is a cause of mastitis.

The SCC is typically determined using infrared spectroscopy in the near-infrared range of 4,000 cm⁻¹ to 9,090 cm⁻¹ (1,100 to 2,500 nm). Other methods for measuring SCC are contemplated.

As indicated above, other properties of milk, which can be combined with the MIR spectrum of the milk to determine the likelihood of conception of a cow, include one or more of fat content (i.e. the proportion of milk, by weight, made up by butterfat), protein content, lactose content, and fatty acid content of milk of the cow. These properties are typically determined using spectroscopy analysis of milk in the mid-infrared range.

Other than MIR spectroscopy, the protein content of milk can also be determined using well established techniques such as the standard Kjeldahl process (Total Kjeldahl Nitrogen (TKN) Analysis) which in effect analyses total nitrogen content in milk. Because TKN analysis does not directly measure protein, the result of total nitrogen is converted into percent protein by multiplying by a factor of 6.38. The conversion factor of 6.38 is specific to milk in that it accounts for the nitrogen content of the average known amino acid composition that is present. Other methods for measuring protein content are contemplated.

Other than MIR spectroscopy, the lactose content of milk can also be determined using polarimetry. To do so, all fat and protein is first removed from the milk, for example, by treatment with sulphuric acid and iodine to form a precipitant of proteins. The remaining solution is filtered to remove precipitant and the optical rotation of the filtered solution (containing lactose) is measured using a polarimeter (Reichert Technologies). Based on the measurement, the number of grams of lactose in the milk can be determined. Other methods may be used, such as high performance liquid chromatography (HPLC) with a Thermo Scientific Dionex Corone Charged Aerosol Detector. Other methods for measuring lactose content are contemplated.

As indicated above, the fatty acid content of milk butterfat can be determined using mid-infrared spectroscopy (Ho P N et al., 24 Apr. 2019, Animal Production Science, https://doi.org/10.1071/AN18532; Soyeurt H et al., 2006, J. Dairy Sci., 89(9): 3690-3695). Other techniques include gas-liquid chromatography (Kilcawley K N and Mannion D T, 2017, “Free Fatty Acid Quantification in Dairy Products”, Chapter 12, http://dx.doi.org/10.5775/intechopen.69596) which is the gold-standard approach. A review of techniques is provided in Amores G and Virto M, 2019, “Total and Free Fatty Acids Analysis in Milk and Dairy Fat”, Separations, 6, 14, doi:10.3390/separations6010014. Typical fatty acids evaluated include butyric acid (C4:0), caproic acid (C6:0), caprylic acid (C8:0), capric acid (C10:0), lauric acid (C12:0), myristic acid (C14:0), palmitic acid (C16:0), margaric acid (C17:0), stearic acid (C18:0), oleic acid (C18:1 c9), arachidic acid (C20:0), total short-chain fatty acids (C1 to C5), total medium-chain fatty acids (C6 to C12), total long-chain fatty acids (C≥14), and de novo fatty acids. Other methods for measuring fatty acid content are contemplated.

In some embodiments, milk of the cow to be tested for likelihood of conception is a milk obtained from the cow before intended insemination of the cow. In some embodiments, the milk is taken from the cow about 18 to 68 days prior to intended insemination.

As indicated above, analysis of the MIR spectrum of the milk of a cow (and in some embodiments also including an analysis of one or more further properties of the milk) may also be combined with an analysis of one or more properties of the cow from which the milk was obtained as a predictor of fertility and conception outcomes. Accordingly, in some embodiments, the method of the first aspect of the present invention further comprises:

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination; and/or

comparing the one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination; and

determining the likelihood of conception upon insemination of the cow on the basis of the comparison,

wherein the first reference for the one or more properties of the cow is derived from a cow or cows which have conceived at first insemination,

wherein the second reference for the one or more properties of the cow is derived from a cow or cows which did not conceive within a previous mating season and had only one insemination event, and

wherein the first reference and/or the second reference for the one or more properties of the cow are not derived from a cow or cows which have conceived following two or more inseminations and which did not conceive but had more than one insemination event at last mating season.

In some embodiments, the one or more properties of the cow may comprise milk yield (MY) on the day of obtaining the milk of the cow, previous lactation (305-day) milk yield, previous lactation (305-day) fat yield, previous lactation (305-day) protein yield, days in milk (DIM) of the cow on the day of obtaining the milk of the cow, days from calving to insemination event (DAI) of the cow, calving age of the cow from a previous insemination, fertility genomic estimated breeding value (GEBV), and genotype of the cow. Other properties of the cow are contemplated provided that they are related to, and contribute to, fertility outcomes in cows. Some of these properties can be measured or accessed on-farm or on-site provided the facility has the necessary resources and previous conception and milk content information of each cow to do so. Otherwise, the information can be accessed from previously collated information which has been generated and stored off-site.

The first reference and second reference for each property of the cow can be determined as described above with respect to properties of milk of the cow.

In some embodiments, the milk yield represents the amount of milk (in kilograms) produced by a cow from a current lactation on the day of herd or individual cow testing. In accordance with standard commercial practices of herd-testing in Australia, this represents milk obtained from a cow at an am and pm milking.

Previous lactation information is commonly determined over a period of 305 days from day 1 to day 305 of the previous lactation period. Milk yield, fat yield and protein yield over the 305 day period can be determined using the methods described above. Yields are typically expressed in kilograms for the 305 day period.

Days in milk (DIM) refers to the number of days the cow has been producing milk in the current lactation period on the day milk samples of the cow or herd were taken for analysis.

Days from calving to insemination event (DAI) refers to the number of days from the current calving to an insemination.

The calving age of a cow is the age at which the cow calved from the last insemination event. The calving age is typically measured in months.

The genotype of a cow refers to the genetic constitution of the cow which is ultimately responsible for determining the characteristics of the cow. The genotype of the cow may be determined by sequencing the whole genome, or a part thereof, of the cow, or by determining variations in the genome DNA sequence which may impart those characteristics. In some embodiments, the genotype may be determined through the identification of single nucleotide polymorphic (SNP) variants present in the genome of the cow. Identification of SNP variants may be determined using known techniques including the use of SNP microarrays including those available from Illumina Inc. (San Diego, Calif., USA) such as the BovineSNP50 Genotyping BeadChip, or via sequencing and analysis of genomic or exomic DNA.

To incorporate genotype data into a prediction model, a genomic relationship matrix (GRM—a matrix estimating the fraction of total DNA that two individual cows share) can first be derived. For example the GRM will be a matrix of size equivalent to the number of genotyped individuals by number of genotyped individuals that each off-diagonal position of the matrix represents. The GRM can be derived using the method of Yang J et al., 2010, Nature Genet., 42(7): 565-569. An example of how genotype data is included in the prediction model is application of a principal component analysis on the GRM, where principal components from the GRM are included as additional predictors. Other methods of incorporation of genotype data are contemplated.

The fertility genomic estimated breeding value (GEBV) is an estimate of the genetic value for fertility of an animal calculated using genotype information of the cow (e.g. genetic marker data such as SNP data) and a known prediction equation of female fertility (i.e. the GEBV is the sum of the number of specified alleles present at a locus multiplied by the effect at that locus).

It has been shown that the MIR spectrum of milk of a cow plays an important role in providing unexpected and improved predictive tools with respect to determining the likelihood of conception of a cow upon insemination. The predictive power of the MIR spectrum can be derived and expressed in a number of ways, and is typically derived by statistical modelling of MIR spectrum values and expressed as a percent or proportion of a correct prediction of pregnant or open cows (defined as sensitivity and specificity, respectively). For example, use of the MIR spectrum predicted a good likelihood of conception upon insemination correctly in testing on data excluded from model development in about 68% to 75% of cows that were classified as having good fertility from the cohort, and predicted a poor likelihood of conception upon insemination correctly in about 57% to 66% of cows that were classified as having poor fertility from the cohort. Other ways in which the predictive power of the MIR spectrum can be derived and expressed would be known in the art and have been summarized in publications such as Parikh R et al., 2008, Indian J. Ophthalm., 56(1): 45-50.

The predictive power of the MIR spectrum may be enhanced further by combining MIR spectrum data with various other properties of milk of the cow, and/or properties of the cow from which the milk was obtained, as defined herein. For example, in some embodiments, the one or more properties may include the MIR spectrum of milk of the cow, somatic cell count of the milk, milk yield (MY) on the day of obtaining the milk, days in milk (DIM) of the cow on the day of obtaining the milk, days from calving to insemination (DAI) of the cow, and calving age of the cow. As set out below in Example 1, this combination of properties predicted a good likelihood of conception upon insemination correctly in about 75% to 81% of cows that were classified as having good fertility from the cohort, and predicted a poor likelihood of conception upon insemination correctly in about 62% to 68% of cows that were classified as having poor fertility from the cohort.

Other combinations of properties are contemplated by the present invention provided they include the MIR spectrum data. For example, another combination includes the MIR spectrum of milk of the cow, somatic cell count of the milk, milk yield (MY) on the day of obtaining the milk, days in milk (DIM) of the cow on the day of obtaining the milk, days from calving to insemination (DAI) of the cow, calving age of the cow from a previous insemination, and previous lactation information.

In a second aspect, the present invention provides a method of determining the likelihood of conception upon insemination of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination, and/or comparing a mid-infrared (MIR) spectrum of milk of the cow with a second reference MIR spectrum, wherein the second reference MIR spectrum is representative of a cow or cows having a poor likelihood of conception upon insemination; and

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination, and/or comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and/or

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination, and/or comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of each comparison,

wherein the first reference MIR spectrum, the first reference for the one or more further properties of the milk, and the first reference for the one or more properties of the cow, are derived from a cow or cows which have conceived at first insemination,

wherein the second reference MIR spectrum, the second reference for the one or more further properties of the milk, and the second reference for the one or more properties of the cow, are derived from a cow or cows which did not conceive within a previous mating season and had only one insemination event, and

wherein the first reference MIR spectrum, the first reference for the one or more further properties of the milk, the first reference for the one or more properties of the cow, the second reference MIR spectrum, the second reference for the one or more further properties of the milk, and the second reference for the one or more properties of the cow, are not derived from a cow or cows which have conceived following two or more inseminations and which did not conceive but had more than one insemination event at last mating season.

As indicated above with respect to the first aspect of the invention, the MIR spectra can be compared using a statistical comparison as described above.

As indicated above, the one or more properties of milk of a cow to be tested, or the one or more properties of the cow itself, are compared to a first reference and/or a second reference for each property. With the exception of MIR spectra, the first reference and second reference for each property derived from the cohort of cows analysed with respect to the present invention is listed in Table 1 (see Example 1 below). For example, the cohort of cows analysed herein established that the first reference with respect to somatic cell count of the milk of the cohort was an average of about 135 cells/ml, and the second reference was an average of about 110 cells/ml. With respect to milk yield (MY) on the day of obtaining the milk of the cows, the first reference was an average of about 27.6 kg/day, and the second reference was an average of about 28.8 kg/day. With respect to DIM, the first reference was an average of about 62.6 days, and the second reference was an average of about 57.9 days. With respect to DAI, the first reference was an average of about 106.3 days and the second reference was an average of about 96.2 days. With respect to the calving age of the cow from a previous insemination, the first reference was an average of about 48.6 months and the second reference was an average of about 48.4 months.

Accordingly, when determining the likelihood of conception of a test cow when the aforementioned properties of milk from the cow (or properties of the cow itself) are compared to the first reference and/or second reference for each property (and when the compared MIR spectrum of the milk of the cow is also taken into consideration), a cow whose collective properties are more consistent with the first reference for each property than with the second reference for each property will have a good likelihood of conception at insemination. Similarly, if the collective properties of the cow are more consistent with the second reference for each property than with the first reference for each property then the cow will be predicted to have a poor likelihood of conception at insemination.

However, it is to be made clear that the data in Table 1 with respect to the first reference and second reference for the properties is reflective of the cohort of cows used in the specific study presented in Example 1 below. It would be appreciated by a person skilled in the art that variations to these references may be observed in other cohorts or indeed if the currently used cohort were expanded to include other cows.

Given that the methods of the aforementioned aspects of the invention enable the identification of a cow having a good likelihood of conception, the cow may subsequently be selected for artificial insemination. Accordingly, in a third aspect the present invention provides a method of selecting a dairy cow for artificial insemination, the method comprising:

comparing a mid-infrared (MIR) spectrum of milk of the cow with a first reference MIR spectrum, wherein the first reference MIR spectrum is representative of a cow or cows having a good likelihood of conception upon insemination; and/or

comparing a mid-infrared (MIR) spectrum of milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

selecting the cow for artificial insemination on the basis of the likelihood of conception,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and/or the second reference MIR spectrum are not derived from a cow or cows which have conceived following two or more inseminations and which did not conceive but had more than one insemination event at last mating season.

The MIR spectra can be compared using a statistical comparison as described above.

As indicated above, analysis of the MIR spectrum of the milk of the cow may also be combined with an analysis of one or more further properties of the milk of the cow in making a decision on whether to select the cow for artificial insemination. Accordingly, in some embodiments, the method of the third aspect of the present invention further comprises:

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination; and/or

comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and

determining the likelihood of conception upon insemination of the cow on the basis of the comparison; and

selecting the cow for artificial insemination on the basis of the likelihood of conception,

wherein the first reference for the one or more further properties of the milk is derived from a cow or cows which have conceived at first insemination,

wherein the second reference for the one or more further properties of the milk is derived from a cow or cows which did not conceive within a previous mating season and had only one insemination event, and

wherein the first reference and/or the second reference for the one or more further properties of the milk are not derived from a cow or cows which have conceived following two or more inseminations and which did not conceive but had more than one insemination event at last mating season.

As indicated above, analysis of the MIR spectrum of the milk of a cow (and in some embodiments also including an analysis of one or more further properties of the milk) may also be combined with an analysis of one or more properties of the cow from which the milk was obtained in making a decision on whether to select the cow for artificial insemination. Accordingly, in some embodiments, the method of the third aspect of the present invention further comprises:

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination; and/or

comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of the comparison; and

selecting the cow for artificial insemination on the basis of the likelihood of conception,

wherein the first reference for the one or more properties of the cow is derived from a cow or cows which have conceived at first insemination,

wherein the second reference for the one or more properties of the cow is derived from a cow or cows which did not conceive within a previous mating season and had only one insemination event, and

wherein the first reference and/or the second reference for the one or more properties of the cow are not derived from a cow or cows which have conceived following two or more inseminations and which did not conceive but had more than one insemination event at last mating season.

In a fourth aspect, the present invention provides a method of selecting a dairy cow for artificial insemination, the method comprising:

comparing a mid-infrared (MIR) spectrum of milk of the cow with a first reference MIR spectrum, wherein the first reference MIR spectrum is representative of a cow or cows having a good likelihood of conception upon insemination, and/or comparing a mid-infrared (MIR) spectrum of milk of the cow with a second reference MIR spectrum, wherein the second reference MIR spectrum is representative of a cow or cows having a poor likelihood of conception upon insemination; and

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination, and/or comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and/or

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination, and/or comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of each comparison; and

selecting the cow for artificial insemination on the basis of the likelihood of conception,

wherein the first reference MIR spectrum, the first reference for the one or more further properties of the milk, and the first reference for the one or more properties of the cow, are derived from a cow or cows which have conceived at first insemination,

wherein the second reference MIR spectrum, the second reference for the one or more further properties of the milk, and the second reference for the one or more properties of the cow, are derived from a cow or cows which did not conceive within a previous mating season and had only one insemination event, and

wherein the first reference MIR spectrum, the first reference for the one or more further properties of the milk, the first reference for the one or more properties of the cow, the second reference MIR spectrum, the second reference for the one or more further properties of the milk, and the second reference for the one or more properties of the cow, are not derived from a cow or cows which have conceived following two or more inseminations and which did not conceive but had more than one insemination event at last mating season.

The MIR spectra can be compared using a statistical comparison as described above.

A cow determined to have a good likelihood or poor likelihood of conception will be a cow which has good fertility or poor fertility, respectively. Therefore, a measure of the likelihood of conception is a measure of fertility status. Accordingly, in a fifth aspect the present invention provides a method of classifying the fertility of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination; and/or

comparing a mid-infrared (MIR) spectrum of milk of the cow with a second reference MIR spectrum, wherein the second reference MIR spectrum is representative of a cow or cows having a poor likelihood of conception upon insemination;

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference MIR spectrum is derived from a cow or cows which have conceived at first insemination,

wherein the second reference MIR spectrum is derived from a cow or cows which did not conceive within a previous mating season and had only one insemination event, and

wherein the first reference MIR spectrum and/or the second reference MIR spectrum are not derived from a cow or cows which have conceived following two or more inseminations and which did not conceive but had more than one insemination event at last mating season.

The MIR spectra can be compared using a statistical comparison as described above.

As indicated above, analysis of the MIR spectrum of the milk of the cow may also be combined with an analysis of one or more further properties of the milk of the cow in classifying the fertility of the cow. Accordingly, in some embodiments, the method of the fifth aspect of the present invention further comprises:

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination; and/or

comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination;

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference for the one or more further properties of the milk is derived from a cow or cows which have conceived at first insemination,

wherein the second reference for the one or more further properties of the milk is derived from a cow or cows which did not conceive within a previous mating season and had only one insemination event, and

wherein the first reference and/or the second reference for the one or more further properties of the milk are not derived from a cow or cows which have conceived following two or more inseminations and which did not conceive but had more than one insemination event at last mating season.

As indicated above, analysis of the MIR spectrum of the milk of a cow (and in some embodiments also including an analysis of one or more further properties of the milk) may also be combined with an analysis of one or more properties of the cow from which the milk was obtained in classifying the fertility of the cow. Accordingly, in some embodiments, the method of the fifth aspect of the present invention further comprises:

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination; and/or

comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination;

determining the likelihood of conception upon insemination of the cow on the basis of the comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference for the one or more properties of the cow is derived from a cow or cows which have conceived at first insemination,

wherein the second reference for the one or more properties of the cow is derived from a cow or cows which did not conceive within a previous mating season and had only one insemination event, and

wherein the first reference and the second reference for the one or more properties of the cow are not derived from a cow or cows which have conceived following two or more inseminations and which did not conceive but had more than one insemination event at last mating season.

In a sixth aspect, the present invention provides a method of classifying the fertility of a dairy cow, the method comprising:

comparing a mid-infrared (MIR) spectrum of milk of the cow with a first reference MIR spectrum, wherein the first reference MIR spectrum is representative of a cow or cows having a good likelihood of conception upon insemination, and/or comparing a mid-infrared (MIR) spectrum of milk of the cow with a second reference MIR spectrum, wherein the second reference MIR spectrum is representative of a cow or cows having a poor likelihood of conception upon insemination; and

comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination, and/or comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and/or

comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination, and/or comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination

determining the likelihood of conception upon insemination of the cow on the basis of each comparison; and

classifying the cow as having good fertility or poor fertility on the basis of the likelihood of conception, wherein a cow having good fertility will have a good likelihood of conception upon insemination, and a cow having poor fertility will have a poor likelihood of conception upon insemination,

wherein the first reference MIR spectrum, the first reference for the one or more further properties of the milk, and the first reference for the one or more properties of the cow, are derived from a cow or cows which have conceived at first insemination,

wherein the second reference MIR spectrum, the second reference for the one or more further properties of the milk, and the second reference for the one or more properties of the cow, are derived from a cow or cows which did not conceive within a previous mating season and had only one insemination event, and

wherein the first reference MIR spectrum, the first reference for the one or more further properties of the milk, the first reference for the one or more properties of the cow, the second reference MIR spectrum, the second reference for the one or more further properties of the milk, and the second reference for the one or more properties of the cow, are not derived from a cow or cows which have conceived following two or more inseminations and which did not conceive but had more than one insemination event at last mating season.

The MIR spectra can be compared using a statistical comparison as described above.

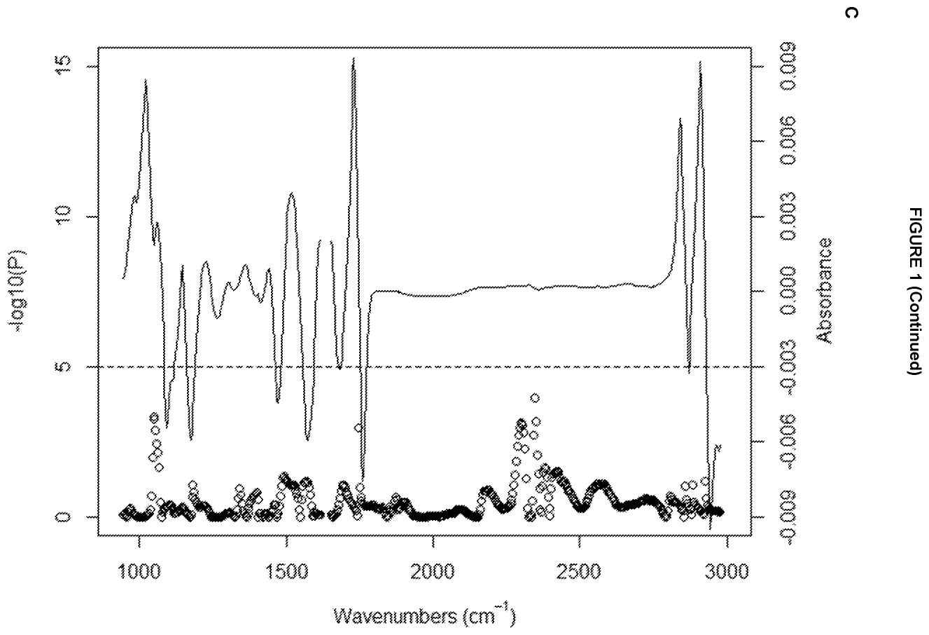

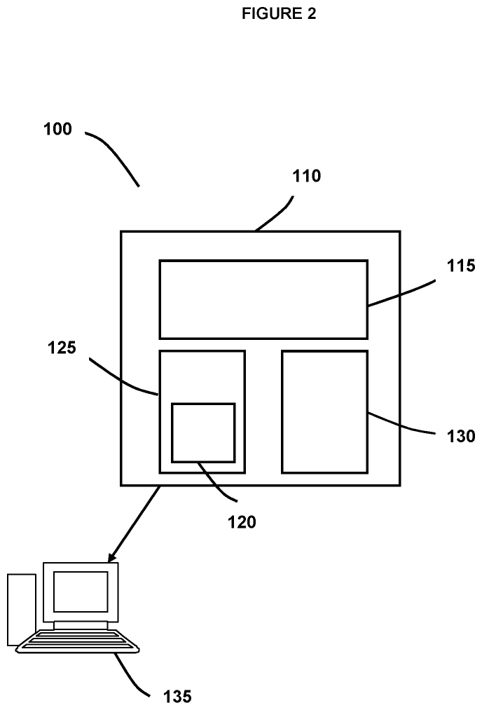

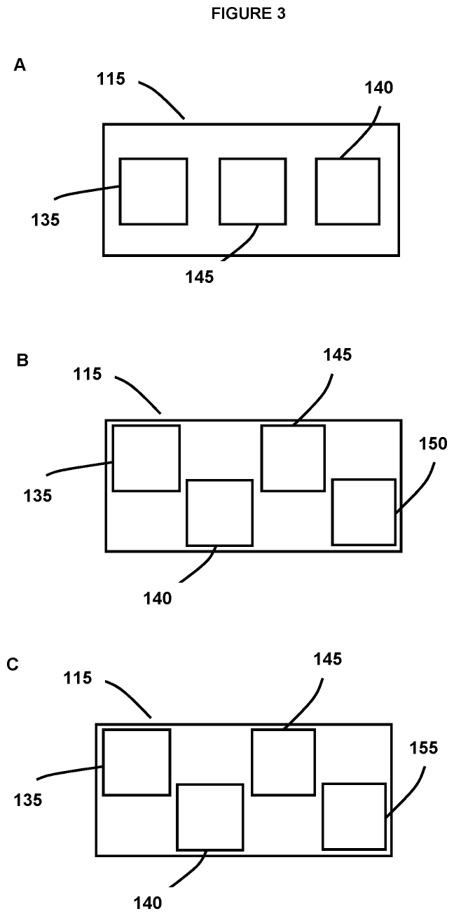

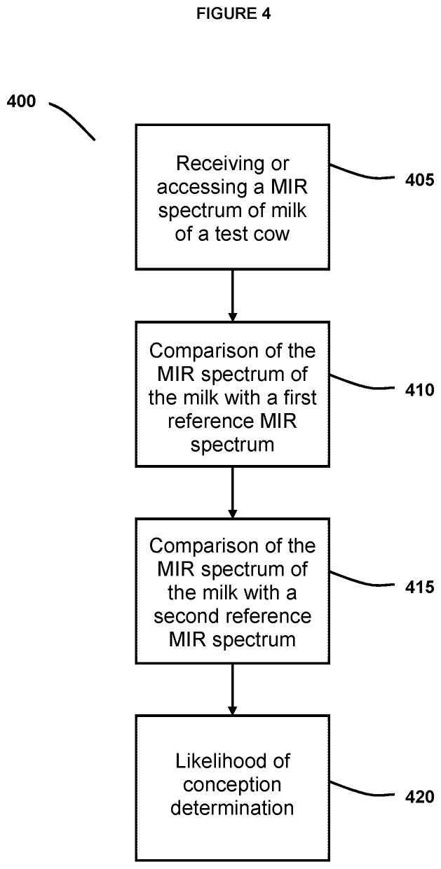

The methods of the aforementioned aspects of the present invention, as described above, can be performed in any manner of means as would be understood by a person skilled in the art. For example, with reference to FIG. 2 there is shown an example system 100 for determining the likelihood of conception upon insemination of a dairy cow according to some aspects of the invention, for selecting a dairy cow for artificial insemination according to some aspects of the invention, and/or for classifying the fertility of a dairy cow according to some aspects of the invention. The system 100 includes a processing unit 110 which stores, receives or accesses information relating to one or more properties of milk obtained from a cow (including the MIR spectrum of the milk), and in some embodiments one or more properties of the cow, including information relating to the first and/or second reference for the one or more properties. The processing unit 110 may include a processor 115 which includes a number of components for processing the information and computing various outputs, or software 120 to carry out these functions. These will be described further with reference to FIGS. 3A to 3C (hardware) and FIGS. 4 to 6 (software). The processing unit 110 also includes a memory 125 for storing data permanently or temporarily and running software 120. A database 130 is included for storing data from the processing unit 110. The processing unit 110 may be connected to a computer 135. The computer 135 may be co-located with the other components of the system 100, or may be located remotely and in data communication with the system 100 over a data network such as a LAN or the internet

As shown in FIGS. 3A to 3C, the processing unit 110 includes a processor 115 which may include dedicated hardware modules or units to carry out hardcoded instructions and provide information to determine the likelihood of conception of a dairy cow upon insemination, select a dairy cow for insemination, or classify the fertility of a dairy cow, respectively. However, it will be appreciated that these modules need not be necessarily implemented in hardware but may be implemented purely in software 120 which is stored on memory 125 and carried out by the processor 115. This will be described with reference to FIGS. 4 to 6.

According to various aspects of the present invention, there is provided a system for determining the likelihood of conception upon insemination of a dairy cow. As shown in FIG. 3A, the processor 115 may include dedicated hardware modules or units including a first comparison unit 135 which compares the MIR spectrum of milk obtained from the cow with a first reference MIR spectrum. There may also be provided a second comparison unit 140 which compares the MIR spectrum of the milk obtained from the cow with a second reference MIR spectrum. The first reference MIR spectrum and second reference MIR spectrum may be stored in memory 125 or on a database 130 of the system 110 and accessed as required by the processor 115. Finally, the processor 115 includes a likelihood of conception determination unit 145 which determines the likelihood of conception upon insemination of the cow on the basis of the comparison (as determined by the comparison units 135 and 140). For this aspect of the invention, and the further aspects described below, the MIR spectral comparisons and likelihood of conception upon insemination determination can be performed by the processor 115 using the statistical comparison algorithms described above. For example, the partial least squares discriminant analysis (PLS-DA).

In some embodiments of the system shown in FIG. 3A, the first comparison unit 135 may also compare one or more further properties of the milk of the cow, and/or one or more properties of the cow from which the milk was obtained, with a first reference for the one or more properties. Furthermore, the second comparison unit 140 may also compare the one or more further properties of the milk of the cow, and/or the one or more properties of the cow, with a second reference for the one or more properties. As indicated above, the first reference and second reference for these one or more properties may be stored in the memory 125 or on the database 130 of the system 110 and accessed as required by the processor 115. The likelihood of conception determination unit 145 of the processor 115 then determines the likelihood of conception upon insemination of the cow on the basis of the collective comparisons (as determined by the comparison units 135 and 140).

According to various aspects of the present invention, there is provided a system for selecting a cow for artificial insemination. FIG. 3B shows the processor 115 including a module for such a selection. As described with reference to FIG. 3A, the processor 115 includes a first comparison unit 135 which compares the MIR spectrum of milk obtained from the cow with a first reference MIR spectrum. There may also be provided, a second comparison unit 140 which compares the MIR spectrum of the milk obtained from the cow with a second reference MIR spectrum. The system according to this embodiment also includes a likelihood of conception determination unit 145. Finally, the processor 115 includes a selection determination unit 150 for selecting a cow for artificial insemination on the basis of the likelihood of conception determined by the likelihood of conception determination unit 145.

In some embodiments of the system shown in FIG. 3B, the first comparison unit 135 may also compare one or more further properties of the milk of the cow, and/or one or more properties of the cow from which the milk was obtained, with a first reference for the one or more properties. Furthermore, the second comparison unit 140 may also compare the one or more further properties of the milk of the cow, and/or the one or more properties of the cow, with a second reference for the one or more properties. The likelihood of conception determination unit 145 of the processor 115 according to this embodiment of the system then determines the likelihood of conception upon insemination of the cow on the basis of the collective comparisons (as determined by the comparison units 135 and 140). The selection determination unit 150 of the system then selects a cow for artificial insemination on the basis of the likelihood of conception determined by the likelihood of conception determination unit 145.

According to various aspects of the present invention, there is provided a system for classifying the fertility of a dairy cow. FIG. 3C shows the processor 115 including a module for such a classification. As described with reference to FIG. 3A, the processor 115 includes a first comparison unit 135 which compares the MIR spectrum of milk obtained from the cow with a first reference MIR spectrum. There may also be provided a second comparison unit 140 which compares the MIR spectrum of the milk obtained from the cow with a second reference MIR spectrum. The system according to this embodiment also includes a likelihood of conception determination unit 145. Finally, the processor 115 includes a classification determination unit 155 for classifying the fertility of the cow on the basis of the likelihood of conception determined by the likelihood of conception determination unit 145.

In some embodiments of the system shown in FIG. 3C, the first comparison unit 135 may also compare one or more further properties of the milk of the cow, and/or one or more properties of the cow from which the milk was obtained, with a first reference for the one or more properties. Furthermore, the second comparison unit 140 may also compare the one or more further properties of the milk of the cow, and/or the one or more properties of the cow, with a second reference for the one or more properties. The likelihood of conception determination unit 145 of the processor 115 according to this embodiment of the system then determines the likelihood of conception upon insemination of the cow on the basis of the collective comparisons (as determined by the comparison units 135 and 140). The classification determination unit 155 of the system then classifies the fertility of the cow on the basis of the likelihood of conception determined by the likelihood of conception determination unit 145.

As indicated above, the hardware modules or units described with reference to FIGS. 3A to 3C may also be implemented in software 120 running in memory 125. FIG. 4 describes a method 400 of the invention for determining the likelihood of conception upon insemination of a dairy cow. At step 405, information relating to the MIR spectrum of milk of the cow, including information relating to a first reference MIR spectrum and/or second reference MIR spectrum, is received or accessed from a processing unit 110 as described in FIG. 2. Control then moves to step 410 where the MIR spectrum of the milk of the cow is compared with the first reference MIR spectrum. This step may be carried out by the processor 115 on the processing unit 110. Control then moves to step 415 where the MIR spectrum of the milk of the cow is compared with the second reference MIR spectrum. This comparison may also be carried out by the processor 115 on the processing unit 110. The first reference MIR spectrum and second reference MIR spectrum may be stored in the database 130 and/or memory 125 of the processing unit 110. Finally, at step 420 the likelihood of conception upon insemination of the cow is determined on the basis of the comparisons determined at steps 410 and 415. The results may then be optionally displayed on a display associated with a personal computer 135.

In some embodiments, at step 405 information relating to one or more further properties of the milk of the cow, and/or one or more properties of the cow from which the milk was obtained, including information relating to a first reference and/or second reference for the one or more properties, is received or accessed from the processing unit 110. In this embodiment, step 410 also compares the one or more properties with the first reference for the one or more properties. Step 415 then compares the one or more properties with the second reference for the one or more properties. Again, the first reference and second reference for the one or more properties may be stored in the database 130 and/or memory 125 of the processing unit 110. Finally, in this embodiment, step 420 determines the likelihood of conception upon insemination of the cow on the basis of the collective comparisons determined at steps 410 and 415.

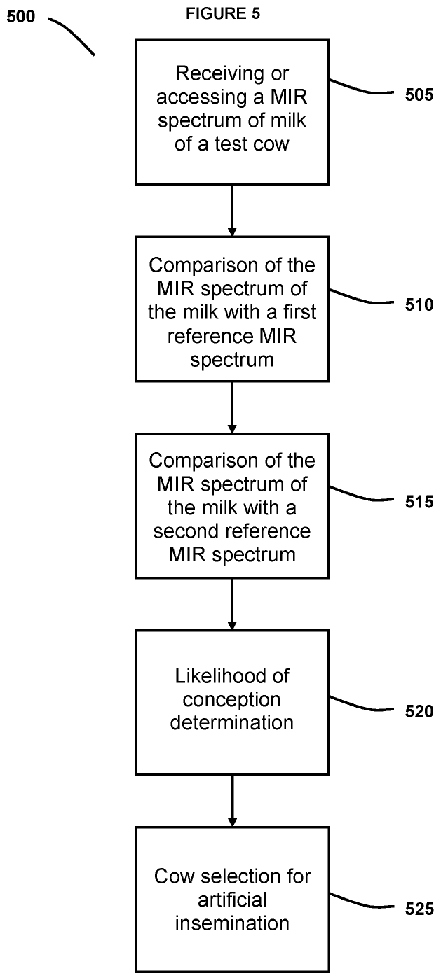

FIG. 5 describes a method 500 of selecting a dairy cow for artificial insemination. At step 505 information relating to the MIR spectrum of milk obtained from the cow, including information relating to a first reference MIR spectrum and/or second reference MIR spectrum, is received or accessed from a processing unit 110 as described in FIG. 2. Control then moves to step 510 where the MIR spectrum of the milk of the cow is compared with the first reference MIR spectrum. This step may be carried out by the processor 115 on the processing unit 110. Control then moves to step 515 where the MIR spectrum of the milk of the cow is compared with the second reference MIR spectrum. This comparison may also be carried out by the processor 115 on the processing unit 110. The first reference MIR spectrum and second reference MIR spectrum may be stored in the database 130 and/or memory 125 of the processing unit 110. Control then moves to step 520 where the likelihood of conception upon insemination of the cow is determined on the basis of the comparisons determined at steps 510 and 515. Finally, at step 525 the cow may be selected for artificial insemination on the basis of the conception likelihood determined in step 520. The results may then be optionally displayed on a display associated with a personal computer 135.

In some embodiments, at step 505 information relating to one or more further properties of the milk of the cow, and/or one or more properties of the cow from which the milk was obtained, including information relating to a first reference and/or second reference for the one or more properties, is received or accessed from the processing unit 110. In this embodiment, step 510 also compares the one or more properties with the first reference for the one or more properties. Step 515 then compares the one or more properties with the second reference for the one or more properties. Again, the first reference and second reference for the one or more properties may be stored in the database 130 and/or memory 125 of the processing unit 110. Step 520 then determines the likelihood of conception upon insemination of the cow on the basis of the collective comparisons determined at steps 510 and 515. Finally, in this embodiment, at step 525 the cow may be selected for artificial insemination on the basis of the conception likelihood determined in step 520.

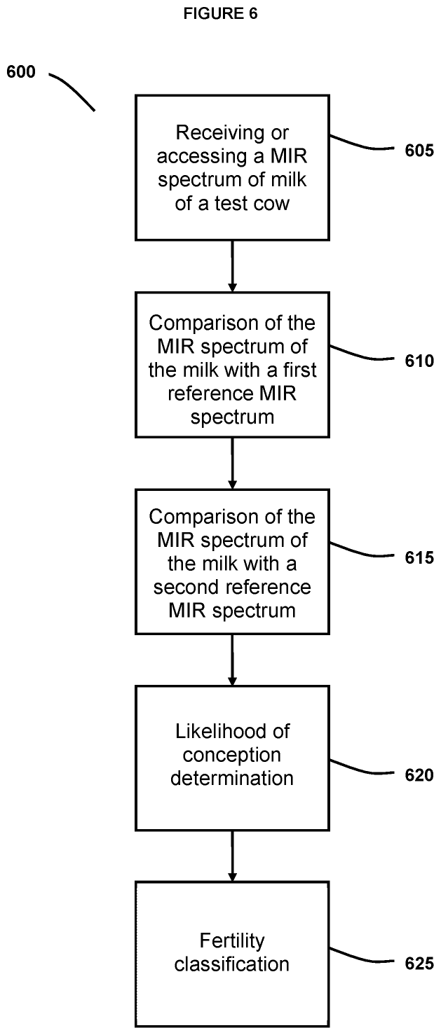

FIG. 6 describes a method 600 of classifying the fertility of a dairy cow. At step 605 information relating to the MIR spectrum of milk of the cow, including information relating to a first reference MIR spectrum and/or second reference MIR spectrum, is received or accessed from a processing unit 110 as described in FIG. 2. Control then moves to step 610 where the MIR spectrum of the milk of the cow is compared with a first reference MIR spectrum. This step may be carried out by the processor 115 on the processing unit 110. Control then moves to step 615 where the MIR spectrum of the milk of the cow is compared with a second reference MIR spectrum. This comparison may also be carried out by the processor 115 on the processing unit 110. The first reference MIR spectrum and second reference MIR spectrum may be stored in the database 130 and/or memory 125 of the processing unit 110. Control then moves to step 620 where the likelihood of conception upon insemination of the cow is determined on the basis of the comparisons determined at steps 610 and 615. Finally, at step 625 the fertility of the cow is classified on the basis of the likelihood of conception determined at step 620. The results may then be optionally displayed on a display associated with a personal computer 135.

In some embodiments, at step 605 information relating to one or more further properties of the milk of the cow, and/or one or more properties of the cow from which the milk was obtained, including information relating to a first reference and/or second reference for the one or more properties, is received or accessed from the processing unit 110. In this embodiment, step 610 also compares the one or more properties with the first reference for the one or more properties. Step 615 then compares the one or more properties with the second reference for the one or more properties. Again, the first reference and second reference for the one or more properties may be stored in the database 130 and/or memory 125 of the processing unit 110. Step 620 then determines the likelihood of conception upon insemination of the cow on the basis of the collective comparisons determined at steps 610 and 615. Finally, in this embodiment, at step 625 the fertility of the cow is classified on the basis of the conception likelihood determined in step 620.

In further aspects, the present invention provides software for use with a computer comprising a processor and memory for storing the software, wherein the software comprises a series of coded instructions for executing a computer process by the processor, wherein the computer process determines any one or more of the following:

(1) determining the likelihood of conception upon insemination of a dairy cow according to a method described herein;

(2) selection of a dairy cow for artificial insemination according to a method described herein; and

(3) classifying the fertility of a dairy cow according to a method described herein.

The computer process may be included in the coded instructions executed in the processing unit and/or comparison and determination units of the device, as described above. The coded instructions may be included in software and they may be transferred via a distribution means. The distribution means may be for example an electric, magnetic or optical means. The distribution means may also be a physical means, such as a memory unit, an optical disc or a telecommunication signal.

As indicated above, a unique herd segregation protocol has been adopted which provides improved accuracy for classifying cows according to their predicted fertility status. The improved accuracy has been achieved based on the segregation of cows for data analysis into extreme groups and excluding data obtained from cows which fall between these two extremes. This segregation has established that the MIR spectrum of milk of a cow is a marker for fertility prediction. Accordingly, the notion of segregation of cows into extreme groups has enabled the identification of reference MIR spectra which can be used to compare with the MIR spectrum of milk of a cow for which fertility status is being determined.

Accordingly, in a further aspect the present invention provides a method of deriving a first reference and/or a second reference for a mid-infrared (MIR) spectrum of milk of a dairy cow, the method comprising:

dividing a cohort of dairy cows into three groups based on previous insemination outcomes, wherein the first group are cows which have conceived at first insemination, wherein the second group are cows which did not conceive within a previous mating season and had only one insemination event, and wherein the third group are cows which have conceived following two or more inseminations and which did not conceive but had more than one insemination event at last mating season;

obtaining or accessing a mid-infrared (MIR) spectrum of milk of each cow of the first group and/or the second group;

comparing the MIR spectrum of the milk of a cow in the first group with the MIR spectrum of the milk of each other cow in the first group to derive a first reference MIR spectrum; and/or

comparing the MIR spectrum of the milk of a cow in the second group with the MIR spectrum of the milk of each other cow in the second group to derive a second reference MIR spectrum,

wherein the first reference MIR spectrum is representative of cows having a good likelihood of conception or good fertility, and wherein the second reference MIR spectrum is representative of cows having a poor likelihood of conception or poor fertility.

In some embodiments of this aspect of the invention, the MIR spectra are compared using a statistical comparison. In some embodiments, the statistical comparison is that of MIR spectral features of each MIR spectrum being compared. In some embodiments, the MIR spectral features are individual wavenumbers of each MIR spectrum.

Deriving a first reference MIR spectrum and/or second reference MIR spectrum may encompass pre-treatment of each MIR spectra obtained for each cow in the first and/or second groups prior to the comparison. For example, as described above spectral regions (2998 to 3998 cm⁻¹, 1615 to 1652 cm⁻¹, and 649 to 925 cm⁻¹) characterized by low signal to noise ratio, which is the consequence of high water absorption, can be removed prior to chemometric analyses. Furthermore, to discard spectra that are potentially outliers, a standardised Mahalanobis distance (which is often known as global H distance) between each spectrum and the cohort average can be calculated. Then, spectra with a global distance greater than 3 can be considered to be outliers and eliminated. Finally, extended multiplicative correction and first order Saviztky-Golay derivative can be applied to the reduced spectra. This pre-treatment process will reduce an original spectrum containing 899 data points to a spectrum with a set of wavenumbers best representing a cow with good fertility or a good likelihood of conception (first reference MIR spectrum), or a cow with poor fertility or a poor likelihood of conception (second reference MIR spectrum). As indicated above, examples of comparisons of reference MIR spectra are shown in FIG. 1.

In some embodiments of this aspect of the invention, the method may further include deriving a first reference and/or a second reference for one or more further properties of the milk of the cow. In this regard, in some embodiments the method further comprises:

obtaining or accessing one or more further properties of the milk of each cow of the first group and/or the second group, wherein the one or more further properties of the milk are related to fertility, and;

comparing the one or more further properties of the milk of a cow in the first group with the one or more further properties of the milk of each other cow in the first group to derive a first reference for the one or more further properties of the milk; and/or

comparing the one or more further properties of the milk a cow in the second group with the one or more further properties of the milk of each other cow in the second group to derive a second reference for the one or more further properties of the milk,

wherein the first reference for the one or more further properties of the milk is representative of cows having a good likelihood of conception or good fertility, and wherein the second reference for the one or more further properties of the milk is representative of cows having a poor likelihood of conception or poor fertility.

In some embodiments, the one or more further properties of the milk comprise somatic cell count (SCC), fat content, protein content, lactose content, and fatty acid content.

In some embodiments of this aspect of the invention, the method may further include deriving a first reference and/or a second reference for one or more properties of a cow from which the milk was obtained. In this regard, in some embodiments the method further comprises:

obtaining or accessing one or more properties of each cow of the first group and/or the second group, wherein the one or more properties of each cow are related to fertility, and;

comparing the one or more properties of a cow in the first group with the one or more properties of each other cow in the first group to derive a first reference for the one or more properties; and/or

comparing the one or more properties of a cow in the second group with the one or more properties of each other cow in the second group to derive a second reference for the one or more properties,

wherein the first reference is representative of cows having a good likelihood of conception or good fertility, and wherein the second reference is representative of cows having a poor likelihood of conception or poor fertility.

The one or more properties of the cow may be those as described above.

The aforementioned method can be applied to any herd or cohort of cows. Once obtained, the first reference and/or second reference for the one or more properties may be stored in a database accessible by users or subscribers. For example, the user or subscriber may be a farmer who wishes to determine the fertility status of one of their cows prior to an intended insemination event. The farmer can obtain a sample of milk from the cow and have one or more properties of the milk determined. The farmer may also obtain one or more properties of the cow from which the milk sample was obtained. The farmer may access the database to compare the one or more properties with the first and/or second reference for each property. Alternatively, the farmer may send the one or more determined properties to a third party who has access to the database to conduct the comparison on their behalf. Alternatively, the farmer may send the milk sample to a commercial testing laboratory, such as TasHerd Pty Ltd (Hadspen, Tasmania, Australia) or Hico Pty Ltd (Maffra, Victoria, Australia), who will determine one or more properties of the milk for subsequent comparison.

In some embodiments, the first reference for a property may be derived from an average value for that property in the cows of the first group. Similarly, the second reference for a property may be derived from an average value for that property in the cows of the second group. Once obtained, the first reference and/or second reference for the one or more properties can be used in the methods, systems and software as described above for determining the likelihood of conception upon insemination of a dairy cow, selecting a dairy cow for insemination, or classifying the fertility of a dairy cow.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications which may be made without departing from the scope of the inventive concept disclosed in this specification. The disclosure also includes all of the steps, features, compositions and compounds referred to, or indicated in this specification, individually or collectively, and any and all combinations of any two or more of the steps or features.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers

It is to be noted that where a range of values is expressed, it will be clearly understood that this range encompasses the upper and lower limits of the range, and all numerical values or sub-ranges in between these limits as if each numerical value and sub-range is explicitly recited. The statement “about X% to Y%” has the same meaning as “about X% to about Y%,” unless indicated otherwise.

The term “about” as used in the specification means approximately or nearly and in the context of a numerical value or range set forth herein is meant to encompass variations of +/−10% or less, +/−5% or less, +/−1% or less, or +/−0.1% or less of and from the numerical value or range recited or claimed.

As used herein, the singular forms “a,” “an,” and “the” may refer to plural articles unless specifically stated otherwise.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

The subject headings used herein are included only for the ease of reference of the reader and should not be used to limit the subject matter found throughout the disclosure or the claims. The subject headings should not be used in construing the scope of the claims or the claim limitations.

The invention is further illustrated in the following examples. The examples are for the purposes of describing particular embodiments only and are not intended to be limiting with respect to the above description.

### EXAMPLE 1

### Classifying the Fertility of Dairy Cows

This first study investigated the potential of milk mid-infrared (MIR) spectra, with or without other variables, for classifying cows of good and poor likelihood of conception upon insemination. Although MIR is routinely used by worldwide milk recording organisations to quantify the concentration of fat, protein, and lactose in milk samples, a number of studies have concluded that the inclusion of MIR spectra did not improve the accuracy of predicting the likelihood of conception to an insemination compared to the use of the same parameters but without MIR spectra.

### Materials and Methods

### Animal Data

Records of insemination and date of calving were available for 8,064 spring-calving cows from 19 commercial dairy herds located in Victoria, Tasmania, and New South Wales of Australia in 2016 and 2017. The cows were between 1^{st} and 6^{th} parity and predominantly Holstein-Friesian (74.3%), but the dataset also included 8.2% purebred Jersey and 17.5% crossbred animals. Other data available included: days in milk (DIM) at herd-test, days from calving to insemination (DAI), age at calving, previous lactation milk yield, milk fat yield, and milk protein yield (all expressed on a 305-day basis), current lactation herd-test day milk yield (MY), fat, protein, and lactose percentages, somatic cell count (SCC), milk and serum fatty acids, β-hydroxybutyrate, urea, fertility genomic estimated breeding value (GEBV), genotype of the cow, and MIR spectra.

Milk fatty acids and blood metabolic profiles were predicted from MIR using the equations developed by Ho P N et al., 2019, supra, and Luke T D et al., 2019, J. Dairy Sci., 102(2): 1747-1760, respectively. Milk production, milk composition, insemination and calving records, fertility GEBV, and 47,162 SNP genotypes (BovineSNP50 BeadChip), edited for the routine genomic evaluations, were obtained from DataGene (https://www.datagene.com.au/).

To incorporate the genotype data into the prediction model, a genomic relationship matrix (GRM—a matrix of 8,604 by 8,604 estimating the fraction of total DNA that two individual cows share) was first derived using the method of Yang J et al., 2010, Nature Genet., 42(7): 565-569. A principal component analysis was then applied on the GRM using the R function prcomp. To determine the optimal numbers of GRM components to be included in the future analyses, a model (i.e., Model 7 as described later) that included MIR spectra, previous lactation 305-d milk yield, milk fat yield, and milk protein yield, current lactation herd-test day milk yield, DIM at herd-test, days from calving to insemination, calving age, and fertility GEBV, was iteratively run with a descending order of size of eigen value. The preliminary analysis showed that the first 84 components (explaining 84.6% of the total variation of the GRM) produced the greatest contribution to the prediction accuracy and thus were used for model development.

### Spectral Data

In this dataset, all cows were milked twice daily in accordance with the standard commercial practices of herd-testing organization in Australia. Milk samples (either am or pm) were collected and sent to TasHerd Pty Ltd (Hadspen, Tasmania, Australia) to be analysed for fat, protein, and lactose concentrations and somatic cell count by Bentley Instruments NexGen Series FTS Combi machine and the corresponding spectra were obtained for this study. Each cow had 2 to 8 records. A recorded spectrum includes 899 data points, with each point representing the absorption of infrared light through the milk sample at a particular wavenumber in the 649 to 3,999 cm⁻¹ region.

### Data Manipulation

The main objective of this study was to examine the potential of MIR spectra alone, and when combined with other on-farm data, for classifying cows of good and poor likelihood of conception upon insemination. Therefore, we first divided the cows in the dataset into three groups as shown in Table 1, including “good” (cows that had conceived at first insemination), “average” cows (cows that had conceived following two or more inseminations and which had not conceived but had had more than one insemination), and “poor” (cows which had not conceived within a previous mating season and had had only one insemination event). The conception was confirmed by a calving in the subsequent year and was coded binarily as 1 (pregnant) and 0 (open). Mating records that resulted in abortions were removed from the data. The conception event was assumed to result from the last recorded insemination.

TABLE 1
Description (mean ± SD) of properties used, besides infrared spectra, to
derive a reference for the properties to classify cows of good, average,
and poor likelihood of conception at first insemination¹

| | Class of likelihood of conception at first insemination | | | |
|---|---|---|---|---|
| | Good | Average | Poor | |
| | (N = 4123) | (N = 2356) | (N = 2618) | P-value² |
| DIM (d) | 62.6 ± 56.9 | 69.0 ± 58.5 | 57.9 ± 49.9 | *** |
| DAI (d) | 106.3 ± 59.2 | 144.4 ± 91.7 | 96.2 ± 49.9 | *** |
| Age at calving (mo) | 48.6 ± 24.6 | 56.8 ± 30.6 | 48.4 ± 24.5 | *** |

Traits of previous lactation (305-d kg)

| | | | | |
|---|---|---|---|---|
| Milk yield | 6901 ± 1734 | 7185 ± 1759 | 7319 ± 1813 | *** |
| Fat yield | 280.5 ± 61.9 | 279.1 ± 61.3 | 293.7 ± 67.1 | *** |
| Protein yield | 236.0 ± 55.8 | 240.8 ± 55.8 | 248.1 ± 59.1 | *** |
| Lactose yield | 324.3 ± 82.0 | 324.8 ± 84.1 | 345.8 ± 84.9 | *** |

Traits of current lactation (per herd-test day)

| | | | | |
|---|---|---|---|---|
| Milk yield (kg/d) | 27.6 ± 7.8 | 28.9 ± 8.6 | 28.8 ± 9.0 | *** |
| Fat (%) | 3.65 ± 0.83 | 3.49 ± 0.82 | 3.76 ± 1.09 | *** |
| Protein (%) | 3.35 ± 0.40 | 3.22 ± 0.43 | 3.28 ± 0.42 | *** |
| Lactose (%) | 5.11 ± 0.19 | 5.10 ± 0.21 | 5.09 ± 0.21 | *** |
| SCC | 135 ± 523 | 166 ± 590 | 110 ± 377 | ** |

Milk fatty acids (g/100 g of milk)

| | | | | |
|---|---|---|---|---|
| C4:0 | 0.096 ± 0.044 | 0.086 ± 0.044 | 0.101 ± 0.051 | *** |
| C6:0 | 0.048 ± 0.027 | 0.042 ± 0.027 | 0.051 ± 0.033 | *** |
| C8:0 | 0.031 ± 0.017 | 0.027 ± 0.016 | 0.033 ± 0.020 | *** |
| C10:0 | 0.066 ± 0.041 | 0.051 ± 0.040 | 0.065 ± 0.049 | *** |
| C12:0 | 0.066 ± 0.049 | 0.056 ± 0.048 | 0.071 ± 0.058 | *** |
| C14:0 | 0.294 ± 0.121 | 0.272 ± 0.121 | 0.031 ± 0.150 | *** |
| C16:0 | 1.250 ± 0.399 | 1.175 ± 0.426 | 1.292 ± 0.459 | *** |
| C17:0 | 0.039 ± 0.007 | 0.038 ± 0.007 | 0.039 ± 0.008 | *** |
| C18:0 | 0.223 ± 0.103 | 0.208 ± 0.109 | 0.229 ± 0.114 | *** |
| 018:1 c9 | 0.659 ± 0.205 | 0.630 ± 0.213 | 0.681 ± 0.227 | *** |
| 020:0 | 0.004 ± 0.002 | 0.004 ± 0.002 | 0.004 ± 0.002 | NS |
| Short-chain FAs | 0.232 ± 0.125 | 0.203 ± 0.125 | 0.248 ± 0.151 | *** |
| Medium-chain FAs | 1.713 ± 0.524 | 1.611 ± 0.548 | 1.771 ± 0.624 | *** |
| Long-chain FAs | 0.885 ± 0.309 | 0.839 ± 0.324 | 0.916 ± 0.349 | *** |
| De novo FAs | 1.256 ± 0.544 | 1.161 ± 0.462 | 1.311 ± 0.551 | *** |

Blood metabolic profiles (mmol/L of blood)

| | | | | |
|---|---|---|---|---|
| Fatty acids | 0.445 ± 0.172 | 0.410 ± 0.184 | 0.484 ± 0.167 | *** |
| 3-hydroxybutyrate | 0.427 ± 0.168 | 0.475 ± 0.156 | 0.382 ± 0.172 | *** |
| Urea | 0.676 ± 0.168 | 0.649 ± 0.182 | 0.692 ± 0.161 | *** |
| Fertility GEBV | 103.5 ± 4.5 | 102.6 ± 4.2 | 103.2 ± 4.6 | *** |

N = number of records,
DIM = days in milk at herd-test,
DAI = days from calving to insemination,
SCC = somatic cell count,
GEBV = genomic estimated breeding value.
¹Good = cows which have conceived at first insemination,
Average = cows which have conceived following two or more inseminations and which did not conceive but had more than one insemination event at last mating season, and
Poor = cows which did not conceive within a previous mating season and had only one insemination event.
²P values obtained from one-way ANOVA tests with pairwise comparisons:
* = P < 0.05,
*** = P < 0.0005,
NS = non-significant (P ≥ 0.05).

It was hypothesized that cows in the “good” and “poor” groups might have significantly different metabolic conditions, and consequently different likelihood to conceive, while the metabolic condition of cows in the “average” group could be similar to that of cows in the other two groups. By focusing on the “good” and “poor” groups, the differences would be magnified and would possibly help improve the predictability of the model. Second, only spectral records obtained before the first insemination were retained, which reduced the data to 6,488 records of 2,897 cows for final analyses. The mean and SD of the number of days between milk sampling for spectral collection and the planned first insemination event were 43.4±25.1. Although there were multiple spectra per cow (i.e., 2.2 on average), we considered each spectrum to be unique because of the large differences in terms of, for example, diet, lactation stage, and management at the time each observation was recorded, which is a common practice in many MIR studies (see for example Soyeurt H et al., 2011, J. Dairy Sci., 94(4): 1657-1667; McParland S et al., 2014, J. Dairy Sci., 97(9): 5863-5871; and van Gastelen S et al., 2018, J. Dairy Sci., 101(6): 5582-5598).

Pre-treatments were also applied to the raw spectra. Firstly, spectral regions (2998 to 3998 cm⁻¹, 1615 to 1652 cm⁻¹, and 649 to 925 cm⁻¹) characterized by low signal to noise ratio, which is the consequence of high water absorption, were removed prior to chemometric analyses (Hewavitharana A K and van Brakel B, 1997, Analyst, 122(7): 701-704). This resulted in 536 wavenumbers available for model development. Secondly, to discard the spectra that are potentially outliers, a standardised Mahalanobis distance (which is often known as global H distance (Shenk J S and Westerhaus M O, 1995, Forage analysis by near infrared spectroscopy. Pages 111-120 in Forages. Vol. II. The Science of Grassland Agriculture. 5th ed. R. F. Barnes, D. A. Miller, and C. J. Nelson, ed., Iowa State University Press, Ames, Iowa)) between each spectrum and the population average was calculated. Then, spectra with a global distance greater than 3 (N=24) were considered to be outliers and eliminated as recommended by Williams P, 2004 (Near-infrared technology: getting the best out of light: a short course in the practical implementation of near-infrared spectroscopy for the user. PDK Projects, Incorporated: Nanaimo, Canada). Finally, extended multiplicative correction (Kohler A et al., 2009, 2.09—Standard Normal Variate, Multiplicative Signal Correction and Extended Multiplicative Signal Correction Preprocessing in Biospectroscopy. Pages 139-162 in Comprehensive Chemometrics. S. D. Brown, R. Tauler, and B. Walczak, ed. Elsevier, Oxford) and first order Saviztky-Golay derivative (Savitzky A and Golay M J, 1964, Analytical Chemistry, 36(8): 1627-1639) were applied to the reduced spectra.

The prediction equations of Ho P N et al., 2019, supra, and Luke T D et al., 2019, supra, were applied on the pre-processed spectra to derive milk fatty acids (C4:0, C6:0, C8:0, C10:0, C12:0, C14:0, C16:0, C17:0, C18:0, C18:1 c9, and C20:0) and the concentrations in sera of fatty acids, β-hydroxybutyrate, and urea, respectively.

### Model Development and Evaluation of Performance

Discriminant models to classify cows that conceived at first insemination or those that did not conceive within the breeding season were developed using partial least squares discriminant analysis (PLS-DA) and implemented with the mixOmics R package of Lê Cao K-A et al., 2011, BMC Bioinformatics, 12(1): 253. PLS-DA is a variant of partial least squares regression when the response variable is categorical, which is used to find the relationship between two matrices. It is one of the most well-known classification methods in chemometrics, metabolomics, and proteomics with an ability to analyze highly collinear data which is often a problem with conventional regression methods, for example, logistic regression (Gromski P S et al., 2015, supra).

The predictors were scaled using an option in the package (i.e. each variable is standardised by dividing itself by the standard deviation). Each model's performance was evaluated in two ways: 10-fold random cross-validation and herd-by-herd external validation. In the 10-fold random cross-validation, the dataset was randomly split into 10 parts that were balanced in terms of the ratio of pregnant and open cows, using the groupdata2 R package (Olsen R L, 2017, Subsetting methods for balanced cross-validation, time series windowing, and general grouping and splitting of data Accessed on: 17-12-2018). One part was reserved for validation, while the remaining data was used for model training. This process was repeated 10 times until each part of the data had been validated once. In the herd-by-herd external validation, the data of a given herd was excluded and used as a validation of the model trained with the data of the other 18 herds. The process continued until every herd had been validated once (i.e., 19 times, as there were 19 herds in this study).

The accuracy of each discriminant model was evaluated by producing the receiver operating characteristic (ROC) curves and calculating the area under the curve (AUC) through the two validation processes described previously. The optimal cut-off value for each test variable was defined as the point where the sum between sensitivity and specificity was at a maximum (i.e., equal weighing of false-positive and false-negative test results), where sensitivity is the proportion of pregnant cows that were correctly classified and specificity is the proportion of open cows that were correctly classified. The PLS-DA method employed in the mixOmics package already uses a prediction threshold based on distances that optimally determine class membership of the samples tested, and therefore, according to Lê Cao K-A et al., 2011, supra, AUC and ROC are not needed to estimate the performance of the model and are provided only as complementary performance measures. The estimated p-values from Wilcoxon tests between the predicted scores of one class versus the other was also obtained, but because they were all statistically significant, they are not reported here.

In this study, twelve models composed of different explanatory variables were tested for their capability in classifying cows of good and poor likelihood of conception (see Table 2). Models 0 and 1 included features that are always available on farms that adhere to the herd-testing program, such as milk production, milk composition, DIM at herd-test, and DAI. These models did not incorporate MIR spectrum data. Models 2 and 3 aimed to compare the additional value of milk fatty acids and blood metabolic profiles versus the MIR spectrum when being incorporated into the basic model, respectively. Fat, protein, and lactose percentages, milk fatty acids, and blood metabolic profiles were removed from Model 3 to create Model 4. Preliminary results showed that adding MIR spectra produced comparable prediction accuracy (Model 4) compared to the model using both MIR-derived traits and the spectra (Model 3), thus MIR-derived traits were not considered in future models. Accordingly, Models 5, 6, and 7 were used to investigate the contribution of adding the fertility GEBV and/or animal genotypes on top of the predictors in Model 4 to the model performance. Model 8 was the same as Model 4 but did not include the previous lactation information. Model 9 only included MIR spectrum data, and models 10 and 11 added in DIM and DAI data (Model 10) and DIM, DAI and SCC data (Model 11) to the MIR spectrum data.

TABLE 2
Predictor properties included in each model for classifying cows of good and poor
likelihood of conception at first insemination

| | | | | | Previous | | | | | | Milk | | | |
|---|---|---|---|---|---|---|---|---|---|---|---|---|---|---|
| | | | | Calving | lactation | | | | | | fatty | | Fertility | |
| Model | MIR | DIM | DAI | age | information | MY | Fat | Protein | Lactose | SCC | acids | MP | GEBV | Genotype |
| 0 | | x | x | x | | x | | x | | | | | | |
| 1 | | x | x | x | x | x | x | x | x | x | | | | |
| 2 | | x | x | x | x | x | x | x | x | x | x | x | | |
| 3 | x | x | x | x | x | x | x | x | x | x | x | x | | |
| 4 | x | x | x | x | x | x | | | | x | | | | |
| 5 | x | x | x | x | x | x | | | | x | | | x | |
| 6 | x | x | x | x | x | x | | | | x | | | | x |
| −7 | x | x | x | x | x | x | | | | x | | | x | x |
| 8 | x | x | x | x | | x | | | | x | | | | |
| 9 | x | | | | | | | | | | | | | |
| 10 | x | x | x | | | | | | | | | | | |
| 11 | x | x | x | | | | | | | x | | | | |

MIR = milk mid-infrared spectroscopy,
DIM = days in milk of a cow at herd-test;
DAI = days from calving to insemination (d);
Previous lactation information = 305-day milk yield (kg), 305-day fat yield (kg), and 305-day protein yield (kg);
MY = milk yield on herd-test day (kg/d),
Fat = fat (%),
Protein = protein (%),
Lactose = lactose (%),
Calving age = age at current calving (month);
SCC = somatic cell count;
Milk fatty acids (g/100 g of milk) = C4:0, C6:0, C8:0, C10:0, C12:0, C14:0, C16:0, C17:0, C18:0, C18:1 c9, and C20:0, predicted by Ho PN et al., 24 Apr. 2019, Animal Production Science, https://doi.org/10.1071/AN18532;
MP = blood metabolic profiles [mmol/L of blood] (fatty acids, β-hydroxybutyrate, urea) predicted by Luke TD et al., 2019, J. Dairy Sci., 102(2): 1747-1760;
Fertility GEBV = genomic estimated breeding values for fertility;
Genotype = first 84 principal components of genomic relationship matrix.

The statistical measures of performance of the twelve models were compared using a one-way ANOVA test in R with pairwise comparisons. Noticeably, in order for the seven models to be developed using PLS-DA and subsequently having statistically fair comparisons, a random noise matrix with dimensions of N×p, where N=536 is the number of wavenumbers in the reduced spectra and p is the number of records of the validation set, was generated from a uniform distribution in the interval 0.0 to 1.0 and multiplied by a very small constant of 10⁻¹⁰. Such a matrix was then used in Model 1 and 2 to represent the spectral wavenumbers. This method has been proposed previously to identify the uninformative MIR wavenumbers by Gottardo P et al., 2016, J. Dairy Sci., 99(10): 7782-7790). All analyses in the present study were performed with R statistical software version 3.4.4 (R Development Core Team, 2018, The GNU Project. The R Project for Statistical Computing. Accessed Nov. 4, 2018. http://www.rproject.org/).

### Results and Discussion

The ability to accurately predict the outcome of an individual insemination event given to a cow (i.e., pregnant versus open) would allow farmers to implement strategies to optimize breeding decisions. For instance, sexed semen could be used to breed cows with a good likelihood of conception, whereas beef semen or semen from bulls of known high genetic merit of fertility could be used for cows predicted with poor likelihood of conception. Additionally, farmers might adjust feeding or management strategies to help predicted “poor” cows improve their physiological conditions and potential probability of conception. In this study we found that MIR data obtained from herd-testing in early lactation can be used to predict cows that are divergent in probability of conception.

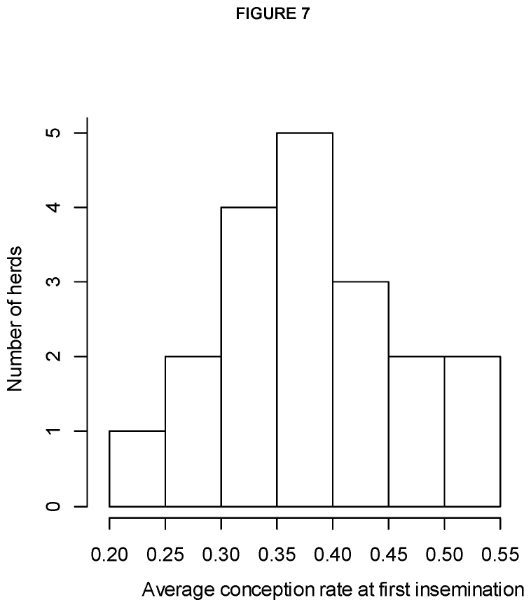

In this study, we found that MIR data alone obtained from herd-testing in early lactation can be used to predict cows that are divergent in probability of conception. In this study, data on 2,987 cows from 19 commercial Australian herds were used to classify cows that contrasted in likelihood of conception to first insemination. The herds were distributed in different regions (mainly in the state of Victoria) to make sure the data were sufficiently representative. This is important because the Australian dairy industry is well recognized to have diverse feeding systems, which range from grazed-pasture to total mixed ration (Dairy Australia, 2016a, Australia's 5 main feeding systems. Dairy Australia. http://www.dairyaustralia.com.au/˜/media/Documents/Animal%20management/Feed%20and%20nutrition/Feeding%20Systems%20latest/Aus%20five%20main%20feeding%20systems.pdf (verified 20 Apr. 2019)). Differences in feeding and genetics have been reported to significantly affect milk composition and thus MIR spectra (Jenkins T C and McGuire M A, 2006, J. Dairy Sci., 89(4): 1302-1310; Gottardo P et al., 2017, Italian J. Anim. Sci., 16(3): 380-389; and Toassini A et al., 2018, Natural Product Res., 33(8): 1085-1091). FIG. 7 presents the conception rate to first service of the herds used in this study. The conception rate ranged from 0.22 to 0.54 with an average of 0.38. These results are comparable with those reported by Dairy Australia 2016b (The InCalf Fertility Data Project 2011, http://www.dairyaustralia.com.au/Animal-management/Fertility/About-I nCalf.aspx (verified 20 Apr. 2019)), where the conception rate to first service ranged between 0.22 and 0.61 with an average of 0.39.

One of the important steps in data editing was splitting the cows into three groups of good, average, and poor fertility, corresponding to those that conceived to one insemination (good), more than one insemination and failed to conceive within a previous mating but being inseminated more than once (average), and failed to conceive within a previous mating season and having only one insemination event (poor). The hypothesis behind this was that cows in the “good” and “poor” groups are more likely to differ in their metabolic status, which would result in different reproductive performance (Oikonomou G et al., 2008, J. Dairy Sci., 91(11): 4323-4332; and Pryce J E et al., 2016, J. Dairy Sci., 99(9): 6855-6873). Such differences in metabolic status are expected to be captured by MIR spectra (Belay T K et al., 2017, J. Dairy Sci., 199(8): 6312-6326; Grelet C et al., 2015, J. Dairy Sci., 98(4): 2150-2160; Pralle R S et al., 2018, J. Dairy Sci., 101(5): 4378-4387; and Luke T D et al., 2019, supra). The metabolic characteristics of the cows in the average group were hypothesized to be similar to those of the other two groups and consequently make them difficult to be differentiated.

As can be seen in Table 1, the means of the predictors for the cows in “good” and “poor” groups seemed to differ from each other more often, whereas “average” cows were similar to those in the other two groups. Cows in the “poor” group produced significantly more milk and had higher yields of fat, protein, and lactose (305-d kg) compared to that of cows in the “good” fertility group (7,319 vs. 6,901, 293.7 vs. 280.5, 248.1 vs. 236.0, and 345.8 vs. 324.3, respectively). Milk, fat, and protein yields of cows in the “average” fertility group were in between the yields in the other two groups. Conversely, the results for several of the other traits in our analysis were not consistent, for example, the “average” cows had higher β-hydroxybutyrate but lower serum fatty acids compared to the “good” cows (0.475 vs. 0.427 and 0.410 vs. 0.445, respectively). The imperfect prediction accuracy of β-hydroxybutyrate (R² ≈ 0.48) and serum fatty acids (R² ≈ 0.61) could be an explanation for this result (Luke T D et al., 2019, supra). Although differences in the means of predictors of cows in the “average” group were statistically significant from those of cows in the “good” and “poor” groups, the pattern was not consistent and therefore makes interpretation difficult. Indeed, we attempted to train the models, using the same explanatory variables, to classify pregnant versus open cows in the entire dataset (i.e. 3 categories instead of 2), and the prediction accuracy was around 50% (data not shown), which can be achieved just by random chance (Chollet F and Allaire J J, 2018, Deep Learning with R. Manning Publications). Accordingly, creating extreme groups to improve model performance was tested and confirmed as providing predictive power in the present study.

Table 3 shows the classification accuracy of the twelve models obtained through 10-fold random cross-validation and the herd-by-herd external validation. The prediction accuracy of all the models obtained through the random cross-validation were consistently higher than that of the herd-by-herd external validation, with the differences in AUC ranging from 0.01 to 0.09. This is understandable because in the first validation approach, the data was first pooled together and then partitioned randomly into 10 parts, without any consideration of cows or their herds. As a result, records from the same herd might have appeared in both the training and validation sets. It should, however, be noted that this is the most common approach used in the majority of MIR prediction studies to evaluate model performance. The small size of reference data is probably the most likely reason for not being able to perform an external validation. A reduction in prediction accuracy in external validation compared to that in random cross-validation has been reported by several authors. Luke T D et al., 2019, supra, observed that the values of coefficients of determination (R²) dropped by 0.07, 0.11, 0.55 for external validation compared to random cross-validation for models predicting serum concentrations of β-hydroxybutyrate, fatty acids, and urea in Australian dairy cows, respectively. McParland S et al., 2012, J. Dairy Sci., 95(12): 7225-7235 indicated that the model for predicting energy balance developed using data from the Scotland's Rural College research farm did not work when applied to the data from the Teagasc Animal and Grassland Research and Innovation Center in Moorepark, Ireland with the correlation coefficient dropping from 0.7 to 0.1. However, the standard deviation of prediction accuracy obtained from herd-by-herd external validation varied more greatly than that obtained from random cross-validation.

TABLE 3
Validation accuracy (mean ± SD) of the partial least squares
discriminant analysis models for classifying cows of good and poor
likelihood of conception at first insemination¹

| | 10-fold random cross-validation | | | | Herd-by-herd external validation | | | |
|---|---|---|---|---|---|---|---|---|
| Model | LV# | Sensitivity | Specificity | AUC | LV# | Sensitivity | Specificity | AUC |
| 0 | 12 | 0.58 ± 0.04 | 0.60 ± 0.07 | 0.60 ± 0.04 | 10 | 0.53 ± 0.19 | 0.57 ± 23 | 0.56 ± 0.09 |
| 1 | 24 | 0.65 ± 0.05 | 0.54 ± 0.04 | 0.66 ± 0.02 | 13 | 0.64 ± 0.15 | 0.61 ± 0.20 | 0.66 ± 0.14 |
| 2 | 24 | 0.72 ± 0.02 | 0.62 ± 0.03 | 0.71 ± 0.02 | 13 | 0.65 ± 0.16 | 0.63 ± 0.20 | 0.68 ± 0.14 |
| 3 | 24 | 0.80 ± 0.02 | 0.68 ± 0.03 | 0.81 ± 0.02 | 10 | 0.73 ± 0.20 | 0.63 ± 0.26 | 0.72 ± 0.13 |
| 4 | 20 | 0.79 ± 0.03 | 0.68 ± 0.03 | 0.80 ± 0.02 | 11 | 0.74 ± 0.20 | 0.62 ± 0.26 | 0.72 ± 0.15 |
| 5 | 22 | 0.81 ± 0.02 | 0.69 ± 0.02 | 0.81 ± 0.02 | 11 | 0.74 ± 0.18 | 0.64 ± 0.23 | 0.74 ± 0.13 |
| 6 | 21 | 0.80 ± 0.02 | 0.71 ± 0.03 | 0.82 ± 0.02 | 13 | 0.75 ± 0.16 | 0.62 ± 0.21 | 0.73 ± 0.12 |
| 7 | 21 | 0.80 ± 0.02 | 0.72 ± 0.03 | 0.83 ± 0.02 | 13 | 0.75 ± 0.16 | 0.66 ± 0.20 | 0.75 ± 0.11 |
| 8 | 24 | 0.81 ± 0.01 | 0.68 ± 0.03 | 0.81 ± 0.02 | 12 | 0.75 ± 0.20 | 0.62 ± 0.26 | 0.72 ± 0.13 |
| 9 | 22 | 0.75 ± 0.01 | 0.66 ± 0.03 | 0.77 ± 0.02 | 11 | 0.68 ± 0.26 | 0.57 ± 0.26 | 0.65 ± 0.10 |
| 10 | 25 | 0.80 ± 0.01 | 0.68 ± 0.03 | 0.81 ± 0.01 | 11 | 0.74 ± 0.24 | 0.62 ± 0.27 | 0.72 ± 0.14 |
| 11 | 25 | 0.80 ± 0.01 | 0.68 ± 0.02 | 0.81 ± 0.02 | 11 | 0.74 ± 0.24 | 0.61 ± 0.27 | 0.72 ± 0.13 |

¹Values with different superscripts within a column are significantly different (P < 0.05);
Good = cows which have conceived at first insemination;
Poor = cows which did not conceive within a previous mating season and had only one insemination event.
LV# = number of latent variables included in the model.
Sensitivity = proportion of pregnant cows that were correctly classified;
Specificity = proportion of open cows that were correctly classified;
AUC = area under the curve of the receiver operating curve.

Interestingly, the average classification accuracy of the best models (Models 7 and 8) in this study remained consistently high even in the herd-by-herd external validation with sensitivity, specificity, and AUC of 0.75, 0.66, and 0.75 on average, respectively, for Model 7, and 0.75, 0.62, and 0.72 on average, respectively, for Model 8. According to \dot{S}imundić A-M, 2009, EJIFCC, 19(4): 203-211, the model diagnostic accuracy is good if the value of AUC is between 0.7 and 0.8.

Using random cross-validation as a reference, the results from our study are higher than that of Shahinfar S et al., 2014, J. Dairy Sci., 97(2): 731-742 and Hempstalk K et al., 2015, J. Dairy Sci., 98(8): 5262-5273. Shahinfar S et al., 2014, supra and Hempstalk K et al., 2015, supra, reported a value of AUC of around 0.67 for predicting the likelihood of conception to any given insemination, which is 0.1 lower than our result of 0.77 for Model 9 (MRI spectrum data alone), 0.16 lower than our result of 0.83 for Model 7, and 0.14 lower than our result of 0.81 for Model 8. The low prediction accuracy in these earlier studies could be due to that fact that they did not create extreme groups of cows (but only considered pregnant versus open cows at any given insemination) as in this study and Grzesiak Wet al., 2010, supra. The imperfect heat detection and unknown effects of other factors such as herd, year, male fertility, abortion, and insemination technician capability were claimed to contribute to such poor results. This could further be complicated by synchronization programs, for example, cows that calved late in the season calving system are often synchronized and timed AI without a need to observed the signs of estrus (Herlihy M M et al., 2011, J. Dairy Sci., 94(9): 4488-4501. Hempstalk K et al., 2015, supra, also concluded that including MIR spectra did not improve prediction accuracy, which disagrees with our findings. Specifically, it is clear that when considering MIR spectrum data alone (Model 9), the average classification accuracy is informative and remains high (sensitivity 0.68 to 0.75, specificity 0.57 to 0.66, and AUC 0.65 to 0.77).

In the current study, the inclusion of milk MIR information either indirectly via milk composition, milk fatty acids or blood metabolic profiles, or directly via MIR wavenumbers, significantly improved the model performance compared to the model including only milk production, milk composition, SCC, DIM at herd-test, DAI, and age at calving. The improvement in prediction accuracy was between 0.02 and 0.15 for both validation methods. The results presented in Table 3 imply that using only basic on-farm information (Model 1) was not sufficient to classify cows into two extreme groups. Adding milk fatty acids and blood metabolic profiles predicted using the MIR equations developed by Ho P N et al., 2019, supra, and Luke T D et al., 2019, supra, raised the classification accuracy by 0.02 to 0.05 (Model 2). Interestingly, we further improved the prediction accuracy of Model 2 by between 0.04 and 0.10 by incorporating the MIR spectra (Model 3), implying that MIR spectra capture variation in fertility beyond milk fatty acids and blood metabolic profiles. Using milk metabolomic or proteomic approaches may elucidate some of these compounds (Goldansaz S A et al., 2017, PLOS ONE, 12(5):e0177675; Ceciliani F et al., 2018, J. Proteomics, 178: 92-106; Xu W et al., 2018, Scientific Reports, 8(1): 15828; and Greenwood S L and Honan M C, 2019, J. Dairy Sci., 102(3): 2796-2806).

The removal of MIR-derived traits from Model 3 did not change prediction accuracy, which means that the useful information obtained from the MIR prediction equations of milk fatty acids, blood metabolic profiles, and milk composition is already included in the MIR spectra. These results agree well with the report of Mineur A, 2017 (Use of MIR spectral data of milk in the detection and prevention of lameness in dairy cows. Master thesis of the Gembloux Agro-Bio Tech (GXABT)—The University of Liège. https://matheo.uliege.be/handle/2268.2/3096. Accessed date: Sep. 1, 2019), who showed that adding MIR-predicted fatty acids and metabolic profiles into a model that already has MIR spectra did not improve the prediction accuracy of lame cows. Grelet C et al., 2015, J. Dairy Sci., 98(4): 2150-2160 stated that using the spectra directly as a reflection of animal health and metabolic status would be a better option than the intermediate traits.

Fertility of dairy cows has been reported to be heritable, with estimates ranging from 0.01 to 0.13 depending on the component trait (Haile-Mariam M et al., 2003, Anim. Sci. (Penicuik, Scotland), 76: 35-42; Liu Z et al., 2008, J. Dairy Sci., 91(11): 4333-4343; Berry D P et al., 2014, Animal 8(s1): 105-121). In Australia, the fertility breeding value includes calving interval, lactation length, calving to first service interval, first service non-return rate, pregnancy rate (Haile-Mariam M et al., 2013, J. Dairy Sci., 96(1): 655-667). The incorporations of fertility GEBV and the animal genotypes (derived from the first 84 principal components of the genomic relationship matrix) would, therefore, be expected to improve the performance of the model. Although the difference was not statistically significant, a 1 to 4% increase in sensitivity, specificity, and AUC was observed in Models 5 to 8 when compared to that in Model 4. Compared to the performance of Model 7, discarding fertility GEBV (Model 5) and animal genotype (Model 6) reduced the prediction accuracy by 0.01 and 0.02, respectively.

Although we have shown that the top models (5 to 8) could correctly classify approximately 74% of cows of good and poor likelihood of conception at first insemination, it is important to explore how the models would perform when applied to a random population, i.e., a population that also includes cows from the average group (Table 2). Accordingly, Model 7 was chosen for this test. Briefly, we repeated the process of herd-by-herd external validation for Model 7 and observed the proportion of correct classification for “good”, “average”, and “poor” groups. While the prediction accuracy remained the same for the “good” and “poor” cows (i.e. 0.75, Table 3), this was only 0.49 for the “average” group. In other words, the model predicted half of the “average” group to be pregnant, while the other half to be open after first insemination. The cows predicted as “poor” needed on average 138 days to have their first service given while this was 112 days for the cows predicted as “good”. While imperfect efficiency of heat detection could partly explain this, negative energy balance may be the most common cause. Butler W R 2003, Livest. Prod. Sci., 83(2-3): 211-218 indicated that negative energy balance suppresses the pulsatility of luteinizing hormone (LH) and reduces the responsiveness of the ovary to LH simulation. Further, during a period of negative energy balance, plasma glucose, insulin, and insulin-like growth factor-I (IGF-I) are reduced (Spicer L et al., 1993, J. Anim. Sci., 71(5): 1323-1241), that consequently shifts postpartum ovarian activity and strongly affects the resumption of the ovarian cycles (Senatore E et al., 1996, Anim. Sci., 62(1): 17-23). Leroy J et al., 2008, Reprod. Domestic Anim., 43(5): 612-622 also reported an inferior oocyte quality in negative-energy-balance cows. Importantly, our finding confirms that the model worked to classify cows of “good” and “poor” fertility but only applied to first insemination, and not to any insemination as presented in Shahinfar S et al., 2014, supra and Hempstalk K et al., 2015, supra.

With the average accuracy (i.e., AUC) obtained through random cross-validation and herd-by-herd external validation of 0.83 and 0.75, respectively (Model 7), and 0.81 and 0.72, respectively (Model 8), these models could be used to rank animals in a herd into high versus low likelihood of conception to first service groups. This ranking can further be refined by combining with other information, for example, serum metabolic profiles derived using the equations of Luke T D et al., 2019, supra, and breeding values. Subsequently, farmers may use this information to decide which semen type to give to those groups of cows, or if any other management actions are needed. Moreover, the models might also be used to generate a large number of fertility traits for cows that have MIR records. The MIR-predicted fertility phenotypes could be used for genomic analyses (Gengler N et al., 2018, ICAR Technical Series No. 23: 221). Lastly, because the number of parameters of a PLS-DA model is large (e.g., 547 for Model 7) they are often not reported to be readily applicable to the readers, the model's application is commonly facilitated through sharing an executable file in which the parameters have been embedded.

### Conclusion

In this study, we have shown that when defining reference values for properties of cows and their milk that are predictive of good or poor conception rates, carefully chosen segregation of cows in populations from which the reference values are derived is vital. These references have established that mid-infrared spectroscopy of milk samples collected in early lactation, either alone or when considered with other on-farm data, can be used to classify cows that conceived at first insemination, and those that did not conceive within the breading season, with reasonably good accuracy. The calibration models were externally validated with reliable results. Such information can be useful in decision support tools to help farmers optimize their breeding decisions. The model can also be used to generate, on a large-scale, fertility phenotypes for genomic evaluation.

### EXAMPLE 2

### Predicting Fertility of Dairy Cows

This objective of this second study was to apply the findings of the first study in Example 1 to develop a tool that can be used to identify cows with a high and low likelihood of conception upon insemination. This study again examined the ability of milk mid-infrared (MIR) spectroscopy and other on-farm data, such as milk yield, milk composition, days in milk, calving age, days in milk at insemination, and somatic cell count, but in a larger cohort of cows, to identify cows that were most or least likely to conceive upon insemination. The tool could be used to provide farmers with a list of animals that might be inseminated with premium semen (i.e., if predicted to have a good likelihood of conception—fertile animals) or those that potentially need a specific breeding or management (i.e., if predicted to have a poor likelihood of conception—sub-fertile animals).

### Materials and Methods

### Animal Data

We followed the same approach as in Example 1, but applied to additional data which was added to the dataset used in Example 1, specifically to address the question of whether the model could be validated in a commercial setting where the outcome of mating is unknown. Between 2016 and 2018 inclusive, commercial farmer records, collected by several milk recording organizations, of insemination date, calving date, DIM at herd-test, days from calving to insemination (DAI), age at calving (i.e., interval between birth date and calving date), herd-test day milk yield (MY), fat, protein, and lactose percentages, SCC, calving season (i.e., spring, summer, autumn, and winter), and milk mid-infrared (MIR) spectroscopy were obtained from DataGene (https://www.datagene.com.au/) for 9,850 lactating cows (33,483 records) from 29 commercial dairy herds located in Victoria, Tasmania, and New South Wales of Australia. The cows were between 1^{st} and 8^{th} parity, with an average parity of 2.9 and consisted of Holstein-Friesian (70.9%), purebred Jersey (5.2%), and crossbred animals (23.9%). In terms of calving season, there were 54.2%, 7.7%, 24.4, and 13.7 calvings in spring, summer, autumn, and winter, respectively.

Information on milk characteristics were obtained from the milk samples (either am or pm) sent to Hico Pty Ltd (Maffra, Victoria, Australia), TasHerd Pty Ltd (Hadspen, Tasmania, Australia) or DairyExpress (Armidale, New South Wales, Australia). The milk composition data included fat, protein, and lactose percentages and somatic cell count analyzed by Bentley Instruments NexGen Series FTS Combi machines and the corresponding spectra were retained for this study. A recorded spectrum includes 899 data points, with each point representing the absorption of infrared light through the milk sample at a particular wavenumber in the 649 to 3,999 cm⁻¹ region.

### Data Manipulation

Because the aim of this study was to predict how likely a cow is going to conceive upon insemination (i.e., a future event), only milk-testing records collected prior to the first insemination were retained. The conception (assumed to result from the last recorded insemination) was confirmed by a calving in the subsequent year and was coded binarily as 1 (pregnant) and 0 (open). The insemination records that resulted in abortions were excluded from the data. Consequently, the final dataset comprised 16,628 records of 7,040 cows. The mean±standard deviation of the number of days between milk sampling and first insemination was −49.8±42.1, while that of DIM at milk-test was 46.7±22.9. Similar to Example 1, although some cows had multiple spectra in the same lactation prior to first insemination (i.e., 2.6 on average), we assumed each spectrum to be unique because of large differences in terms of diet, lactation stage, and management etc. at the time each observation was recorded, which is a common practice in MIR studies (Soyeurt H et al., 2011, supra, 94(4): 1657-1667; McParland S et al., 2014, supra, 97(9): 5863-5871; van Gastelen S et al., 2018, supra, 101(6): 5582-5598). Indeed, we tested the models on the dataset where a unique spectrum per cow was randomly retained and comparable prediction accuracies were obtained compared with multiple spectra per cow.

In terms of the spectral pre-treatment, different mathematical methods were used as recommended by De Marchi M et al., 2014, J. Dairy Sci., 97(3): 1171-1186. Firstly, the noisy regions characterized by a low signal to noise ratio, which is the consequence of a high water absorption (1615 to 1652 cm⁻¹ and 649 to 925 cm⁻¹) and the non-informative region (2998 to 3998 cm⁻¹) were removed. Secondly, to discard the spectra that are potentially outliers, a standardised Mahalanobis distance (i.e., global H distance (Shenk J S and Westerhaus M O, 1995, supra)) between each spectrum and the population average was calculated. Then, the spectra with a global distance greater than 3 (n=36) were considered outliers and eliminated. Lastly, extended multiplicative correction (Kohler A et al., 2009, supra) and first order Saviztky-Golay derivative (Savitzky A and Golay M J, 1964, supra) were applied to the reduced spectra. A final spectrum used for model development consisted of 536 wavenumbers.

As milk samples were analyzed by different machines, some differences in spectral response might be expected. In this context, analysis of identical milk samples is often recommended to standardize each machine and to overcome instrument-to-instrument variations (Grelet C et al., 2017, J. Dairy Sci., 100(10): 7910-7921). Unfortunately, this was not possible in the current study because reference samples were not available. Bonfatti V et al., 2017, J. Dairy Sci., 100(3): 2032-2041, developed an alternative method to be applied retrospectively when reference samples are absent and showed promising results. Our preliminary analysis, however, showed that the spectra corrected using the Bonfatti retrospective method produced comparable prediction accuracies with the use of unstandardized spectra and therefore the results presented in this study were based on the unstandardized spectra.

### Model Development and Evaluation

To develop the prediction models, we followed the methodology of Example 1, by first assigning cows in the dataset into “good”, “average”, and “poor” groups based on each cow's fertility status which corresponds to 1) conception to first insemination (“good”), 2) conception after two or more inseminations and where the cow did not conceive, but where the number of inseminations was>1 (“average”), and 3) no conception event recorded and only one insemination (“poor”). The corresponding proportions of records in each category were 42.1%, 47.2%, and 10.7% for “good”, “average”, and “poor”, respectively. The hypothesis was that “good” and “poor” fertility cows might have significantly different metabolic conditions, and consequently have a different likelihood of conception, while the metabolic condition of “average” fertility cows could be similar to that of cows in either of the other two groups. Thus by focusing on the extreme data that includes only “good” and “poor” groups, the differences would be magnified and this we hypothesized would improve the prediction accuracy (see Example 1). In this study, the term “extreme” refers to the extreme cows in terms of hypothesized metabolic conditions, but not limited to others, that subsequently affects the likelihood of conception of a cow.

Then, using a model that was developed on the training set which included only “good” and “poor” fertility cows, we applied to a separate validation set with all cows present in each herd, i.e., all three groups of cows. Although there were 29 herds in the dataset, some herds had more than one year of records, and here we assumed that each herd-year was unique (i.e., 39 herd-years). Accordingly, the training and validation sets were created as follows: for each round, the data of a given herd-year was excluded and used as a validation of the model trained with the data of the other 38 herd-years and this process was continued until every herd-year had been validated once (i.e., 39 times). The size of each herd-year set varied from 55 to 1447 with an average of 423 records. We also tested the models developed using records of the herds that were completely independent of the herd being validated and this produced comparable prediction accuracy to our assumption of unique herd-year. This was done to make sure that there is not a carryover effect of cows in the same herd from one year to the next.

Thirdly, the outcomes of the model were extracted for further analyses. For each cow or record, the model generates the predicted probabilities of being pregnant (1) and open (0) in a numerical scale with their sum being one. On the one hand, the model uses this information to predict if a cow pregnant (if the probability of 1>the probability of 0) or open (if the probability of 1<the probability of 0). On the other hand, the probability could be interpreted as how certain the model is in its prediction (Delhez P et al., 2020, J. Dairy Sci., 103(7): 6258-6270). For example, if the predicted probabilities of cow A and cow B to be pregnant and open are 0.51 and 0.49 and 0.9 and 0.1, respectively, then the model will assign both cows a value of 1 (i.e., pregnant). However, having a probability of 0.9 for cow B implies that the model is more certain about its prediction compared to that of cow A with the probability of 0.51. In other words, the higher the probability the more confident the model is about its prediction and thus in theory has a higher chance to be correct. As a result, we extracted the predictions, not only in classes (1 and 0), but also the corresponding probabilities. Finally, the predicted values were ranked by their probability and selected in varying proportions calculated as percentages (from 10 to 40%) times the total number of records (cows) in that herd, starting from the top of the list (i.e., highest confidence). The prediction accuracy was then calculated as the proportion of records in the selected data to be truly pregnant or open. For example, if one wishes to identify 10% of cows that are potentially failing to get pregnant to first insemination in a herd of 1000 cows, 100 cows should be selected from the predicted list and the prediction accuracy is simply a count of the number of truly open cows in that 100 selected cows.

In this study, three models composed of different explanatory variables were tested for their capability in identifying cows of good and poor likelihood of conception (Table 4). Model 1 included features that are readily available on farms participating in milk recording, such as milk production, milk composition, SCC, and days from calving to insemination. Days in milk and age at calving were incorporated into model 1 to form model 2; these data may not be directly available from milk recording organizations and if that is true, they are generally available from over-arching data management organizations, for example, DataGene Ltd. (https://datagene.com.au/) in Australia. In model 3, MIR was added to model 2, but at the same time milk composition was removed, because the results in Example 1 indicated that the model with MIR and milk composition produced comparable prediction accuracy to that which included only MIR. The explanation was that the information in milk composition is already contained in MIR. The third model is expected to be applicable mainly by herd-testing centres with a modern MIR machine that can store spectral data.

The prediction models were developed using partial least squares discriminant analysis (PLS-DA) and implemented with the mixOmics R package of Lê Cao K-A et al., 2011, supra. The predictors were scaled using a built-in option in the package (i.e., each variable is standardised by dividing itself by the standard deviation). In order for the three models to be developed using PLS-DA and subsequently having statistically fair comparisons, a random noise matrix with dimensions of N×p, where N=536 is the number of wavenumbers in the reduced spectra and p is the number of records of the validation set, was generated from a uniform distribution in the interval 0.0 to 1.0 and multiplied by a very small constant of 10⁻¹⁰. The matrix was then used in models 1 and 2 to represent the spectral wavenumbers.

All analyses in the present study were performed using R statistical software version 3.6.1 (R Development Core Team, 2020, The GNU Project. The R Project for Statistical Computing. Accessed Jan. 4, 2020. http://www.rproject.org/).

TABLE 4
Explanatory variables included in each model for
predicting the likelihood of conception to first insemination

| Model | MIR | DIM | Calving age | DAI | Calving season | MY | Fat | Protein | Lactose | SCC |
|---|---|---|---|---|---|---|---|---|---|---|
| 1 | | | | x | x | x | x | x | x | x |
| 2 | | x | x | x | x | x | x | x | x | x |
| 3 | x | x | x | x | x | x | | | | x |

MIR = milk mid-infrared spectroscopy,
DIM = days in milk at herd-test;
DAI = days from calving to insemination (d);
MY = milk yield on herd-test day (kg/d),
Fat = fat (%),
Protein = protein (%),
Lactose = lactose (%),
Calving age = age at current calving (month);
Calving season = spring, summer, autumn, or winter;
SCC = somatic cell count.

### Results and Discussion

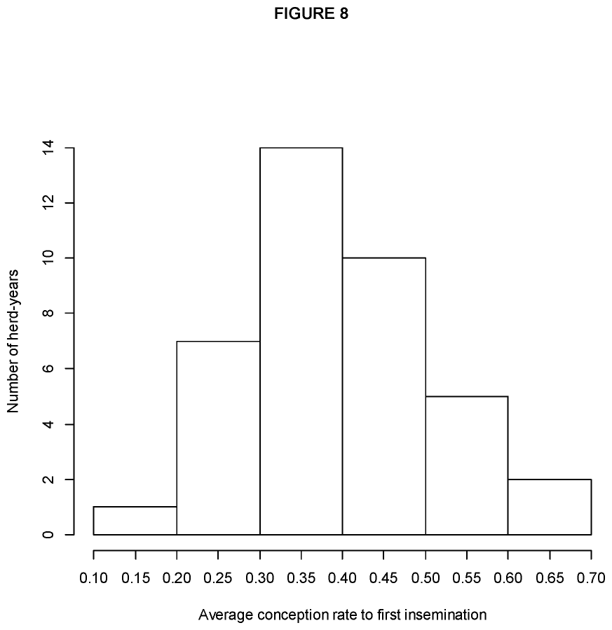

The herd-year mean conception rate to first insemination in the current dataset varied between 0.13 and 0.65 with an average of 0.39 (FIG. 8), which is slightly more variable compared to the report of Dairy Australia, 2011, The InCalf Fertility Data Project 2011. http://www.dairyaustralia.com.au/Animal-management/Fertility/About-InCalf.aspx (verified 21 Nov. 2019), where the mean herd-year conception rate to first insemination ranged between 0.22 and 0.61 with an average of 0.39. Having such variation in herd-level fertility implies that many farmers struggle to get their cows back in-calf postpartum. This is of concern, as good fertility is fundamental in seasonal calving systems to maintain a compact calving period and to match the high energy requirements of the early lactation cow to peak pasture growth rate Armstrong D P et al., 2010, Anim. Prod. Sci., 50(6): 363-370; Shalloo L et al., 2014, Animal, 8(Supplements1): 222-231).

It is worth noting that in Australia, many farmers have moved from seasonal to split or year-round calving systems, largely to accommodate poor fertility. According to the reproductive database from NatSCAN (a national fertility monitoring project in Australia) the percentages of herds with seasonal, split, and year-round calving patterns were 86%, 8%, and 6% in 1997 while in 2016 they were 30%, 47%, 23%, respectively (Ee Cheng Ooi, personal communication, 2020).

Fertility breeding values have been incorporated into the national selection indices of many countries worldwide to help farmers improve the fertility of their herds (Cole J B and VanRaden P M, 2018, J. Dairy Sci., 101(4): 3686-3701). In addition, precision dairy management technologies are increasingly being used to help farmers improve the management of their cows, such as monitoring cow's health and behaviour or detection of estrus and diseases (Bell M J and Tzimiropoulos G, 2018, Frontiers in Sustainable Food Systems, 2(31); Eckelkamp E A and Bewley J M, 2019, J. Dairy Sci., 103(2): 1566-1582).

This study indicates (and confirms the outcome of the study in Example 1) that data collected from a routine milk-test in early lactation could be used to detect cows that potentially have difficulty in getting pregnant to first insemination with promising accuracy. This information could complement other management strategies and evaluating the value of combining sensor and MIR predictions is an area for future research. Another opportunity is prediction of phenotypes when genomic and phenomic information is combined, noting that in Example 1 we found limited advantage with adding fertility EBVs to MIR information in predicting the likelihood of conception to first service. This is perhaps unsurprising, as fertility is well known to be a low heritability trait.

The prediction accuracies of the three models used to identify cows that were most and least likely to conceive to first insemination are presented in Tables 5 and 7, respectively, while Table 6 includes the proportion of cows predicted to conceive to first insemination but actually conceived following two inseminations. The results are reported in proportions of selected cows, varying from 5 to 40% of the cows present in a herd-year. Generally, when more cows are selected, i.e., descending confidence, the accuracy would be reduced. It was shown that selecting 10% of cows with the highest confidence of prediction produced optimal accuracy.

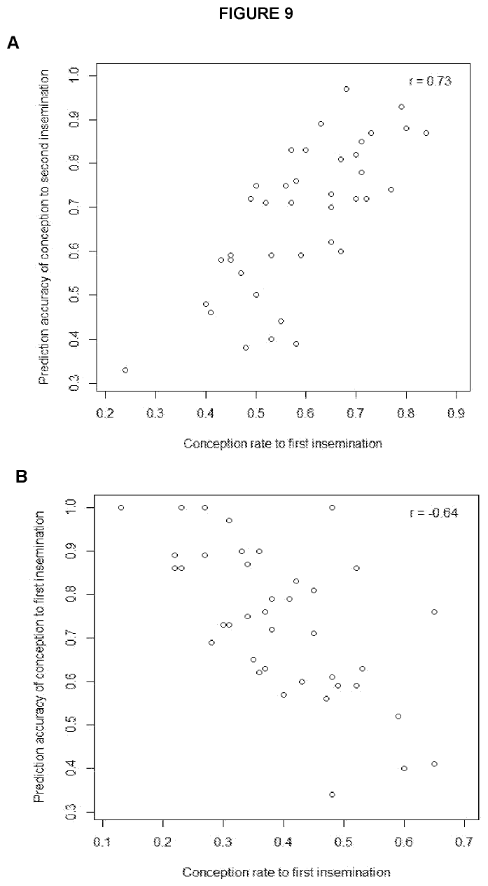

There was considerable variation in prediction accuracy across herd-years with a standard deviation of around 0.16. Interestingly, FIGS. 9A and 9B imply that when attempting to predict cows that had the least likelihood of conception to first and second insemination, the model seemed to perform well on the poor, but less informatively on the high fertility herds. The opposite pattern was observed when using the models for predicting the cows that were most likely to conceive to first insemination, i.e., good performance on the high fertility herds and vice versa.

The correlations between the model's accuracy for predicting cows that failed to first insemination and cows that conceived to second insemination and observed herd-year mean conception rate to first insemination were −0.64 and 0.73, respectively. We also tested the performance of the models developed using two separate datasets based on their fertility level, i.e., high and low fertility, but the same outcome was observed.

TABLE 5
Accuracy of the models for identifying cows with
good likelihood of conception to first insemination¹

| | Model 1 | | Model 2 | | Model 3 | |
|---|---|---|---|---|---|---|
| Proportion | Accuracy | SD | Accuracy | SD | Accuracy | SD |
| 5 | 0.46 | 0.19 | 0.46 | 0.21 | 0.49 | 0.18 |
| 10 | 0.44 | 0.15 | 0.45 | 0.17 | 0.48 | 0.17 |
| 15 | 0.44 | 0.16 | 0.45 | 0.14 | 0.47 | 0.17 |
| 20 | 0.44 | 0.16 | 0.45 | 0.15 | 0.47 | 0.15 |
| 25 | 0.44 | 0.15 | 0.45 | 0.15 | 0.47 | 0.15 |
| 30 | 0.43 | 0.14 | 0.44 | 0.14 | 0.46 | 0.15 |
| 35 | 0.43 | 0.14 | 0.43 | 0.14 | 0.46 | 0.15 |
| 40 | 0.42 | 0.14 | 0.42 | 0.14 | 0.46 | 0.14 |

Proportion = proportion of cows to be selected.
Accuracy = proportion of cows that were correctly predicted as open.
SD = standard deviation.
¹See Table 4 for model descriptions.

TABLE 6
Accuracy of the models for identifying cows with good
likelihood of conception to second insemination¹

| | Model 1 | | Model 2 | | Model 3 | |
|---|---|---|---|---|---|---|
| Proportion | Accuracy | SD | Accuracy | SD | Accuracy | SD |
| 5 | 0.63 | 0.22 | 0.70 | 0.17 | 0.70 | 0.18 |
| 10 | 0.62 | 0.17 | 0.65 | 0.16 | 0.69 | 0.16 |
| 15 | 0.62 | 0.15 | 0.65 | 0.16 | 0.68 | 0.14 |
| 20 | 0.62 | 0.15 | 0.63 | 0.15 | 0.68 | 0.15 |
| 25 | 0.62 | 0.15 | 0.63 | 0.15 | 0.67 | 0.15 |
| 30 | 0.61 | 0.14 | 0.62 | 0.15 | 0.67 | 0.15 |
| 35 | 0.61 | 0.14 | 0.61 | 0.14 | 0.67 | 0.15 |
| 40 | 0.60 | 0.14 | 0.61 | 0.14 | 0.67 | 0.15 |

Proportion = proportion of cows to be selected.
Accuracy = proportion of cows that were correctly predicted as open.
SD = standard deviation.
¹See Table 4 for model descriptions.

TABLE 7
Accuracy of the models for identifying cows
with the poor likelihood of conception to first insemination¹

| | Model 1 | | Model 2 | | Model 3 | |
|---|---|---|---|---|---|---|
| Proportion | Accuracy | SD | Accuracy | SD | Accuracy | SD |
| 5 | 0.66 | 0.19 | 0.69 | 0.18 | 0.76 | 0.17 |
| 10 | 0.64 | 0.16 | 0.67 | 0.15 | 0.76 | 0.15 |
| 15 | 0.63 | 0.14 | 0.65 | 0.14 | 0.72 | 0.14 |
| 20 | 0.62 | 0.13 | 0.64 | 0.14 | 0.71 | 0.13 |
| 25 | 0.61 | 0.14 | 0.63 | 0.13 | 0.69 | 0.13 |
| 30 | 0.60 | 0.14 | 0.62 | 0.13 | 0.68 | 0.12 |
| 35 | 0.60 | 0.13 | 0.61 | 0.13 | 0.66 | 0.11 |
| 40 | 0.60 | 0.13 | 0.60 | 0.12 | 0.66 | 0.12 |

Proportion = proportion of cows to be selected.
Accuracy = proportion of cows that were correctly predicted as open.
SD = standard deviation.
¹See Table 4 for model descriptions.

Compared to model 1, the additions of days in milk and calving age (model 2) only improved the prediction accuracy between 0.01 and 0.03. This implies that the important information associated with the fertility status of the cow is already included in the milk characteristics. Indeed, milk composition and MIR spectra have been used to predict various indicators of fertility such as energy balance (Friggens N C et al., 2007, J. Dairy Sci., 90(12): 5453-5467; McParland S et al., 2011, J. Dairy Sci., 94(7): 3651-3661; Ho P N et al., 2020, Anim. Prod. Sci., 60(1): 164-168), and serum metabolic profiles (Grelet C et al., 2018, Animal, 13(3): 649-658; Pralle R S et al., 2018, J. Dairy Sci., 101(5): 4378-4387; Luke T D et al., 2019, J. Dairy Sci., 101(2): 1747-1760). Consistent with the results in the study in Example 1, this study also shows that the use of MIR spectra improved the prediction accuracy beyond the use of milk composition with a difference ranging between 0.06 to 0.1. This is because milk fat, protein and lactose percentages are predicted from MIR spectra (De Marchi M et al., 2014, supra). Moreover, it also implies that MIR spectra contain other information related to the fertility status of the animal which might be further elucidated using metabolomics (Phillips K M et al., 2018, Scientific Reports, 8(1): 13196), proteomics (Koh Y Q et al., 2018, J. Dairy Sci., 101(7): 6462-6473), or genome-wide association studies (Wang Q and Bovenhuis H, 2018, J. Dairy Sci., 101(3): 2260-2272; Benedet A et al., 2019, J. Dairy Sci., 102(8): 7189-7203). In terms of a practical application, these results mean that MIR was of primary importance in prediction of fertility of dairy cows. As a result, the remaining discussion of this paper will be based on the results obtained for model 3, which was the most predictive one.

Irrespective of the proportions, the accuracy of the model for predicting cows that conceived to first insemination was around 0.48. However, when the same model was used to predict cows that conceived following two inseminations, the accuracy increased substantially (˜0.69). This is interesting because the model was initially trained, or designed, to predict cows that conceived to first insemination, but in the selected predictions only around 48% of them were correct and around 69% of them conceived following 2 inseminations. We suggest that this result occurred because the model picked up the truly “good” fertility cows based on some biomarkers contained in the MIR spectra, which may not be properly represented in the current fertility phenotype (i.e., pregnant versus open). While it is plausible to consider cows that conceived to first insemination to be fertile, assigning cows that failed to conceive to first insemination, but conceived following two inseminations to a sub-fertile group might not be completely appropriate. Some cows in the sub-fertile group might actually be fertile and they could just be unlucky, for example, management errors, such as inseminating too early after calving, or inseminated at an inappropriate time. Multiple factors ranging from the cow's physiology (e.g., milk production, body condition, energy balance, parity, health status) to management (e.g., year, season or time of insemination, semen quality, ability of the technician) have been shown to affect conception rate (Walsh S W et al., 2011, Anim. Reprod. Sci., 123(3): 127-138).

The impact of environment on conception rate to first service, however, is larger compared to the later services (Bormann et al., 2006). So, the definition of fertile cows could be extended to cover those that conceived following two inseminations. Further, six week in-calf rate is a common indicator to evaluate the efficiency of a reproductive program in Australian dairy industry (Dairy Australia 2017, InCalf Book 2nd Edition: https://www.dairyaustralia.com.au/-/media/dairyaustralia/documents/farm/animal-care/fertility/incalf-resources/2017/incalffordairyfarmers2017_webindexed.pdf?la=en&hash=3460E0F31A6F2947D27EBDAA0AD0E2BCC5322316 (verified 21 Nov. 2019)) and in the dataset used in the present study most cows achieved this after two inseminations. We attempted to study this by comparing the difference in spectra between the three groups of cows as defined in Example 1: “good” (cows that conceived to first insemination), “average” (cows that had conceived following two or more inseminations and which had not conceived but had had more than one insemination), and “poor” (cows which had not conceived within a previous mating season and had had only one insemination event) and the results show that the spectra of the “average” cows were very similar to those of “good” cows (FIG. 1C) but more different from the “poor” cows (FIG. 1B). As previously hypothesized in Example 1, the spectra of the “good” and “poor” cows were significantly different (FIG. 1A). Although this result is interesting, further studies should explore what is behind these peaks of spectral differences and to what extent they are related to fertility and again deeper analyses such as metabolomics, proteomics, or gene mapping could play an important role here. If we can find true bio-markers underlying fertility, the accuracy of predicting the fertility of dairy cows might be further improved compared to using the outcomes of current fertility phenotypes. Nevertheless, our approach is applicable in a practical context, where chance plays a role.

When the model was used to predict cows with the least likelihood of conception to first insemination, the accuracy was promising and reached 0.76 on average at 10%, which can be defined as a good prediction (Šimundić A-M, 2009, EJIFCC, 19(4): 203-211). To the best of our knowledge, this type of tool would be unique in the Australian dairy industry. Australian dairy farmers usually make decisions on, for example, which type of semen they inseminate cows based on genetic merit or production level in the previous lactation. It is expected that the model could be used to provide farmers with a list of cows that potentially need special care, or a feeding regime to improve their chances of getting pregnant. It should be noted that the model can perform predictions with data collected as early as around 26 days post-calving, thus farmers would have 8 weeks to act, given the average time from calving to first insemination is 85 days for Australian dairy herds (Haile-Mariam M et al., 2003, Anim. Sci., 76(1): 35-42). Finally, a prediction accuracy for cows that conceived to second insemination of 0.69 is promising, but more studies are needed to confirm the appropriateness of categorizing cows that conceived to first and second insemination as fertile.

### Conclusion

We have successfully developed and tested various models for identifying cows that were most and least likely to conceive to first and second insemination using milk mid-infrared spectra and other on-farm data collected in early lactation with promising accuracy. The most predictive model, including milk yield, MIR, DIM, calving age, DIM at insemination and SCC correctly identified the 10% of cows that were most likely to conceive to first and second insemination and those that were least likely to conceive first insemination with an accuracy of 0.48, 0.69, and 0.76, respectively.

1. A method of determining the likelihood of conception upon insemination of a dairy cow, the method comprising:
comparing a mid-infrared (MIR) spectrum of milk of the cow with a first reference MIR spectrum, wherein the first reference MIR spectrum is representative of a cow or cows having a good likelihood of conception upon insemination; and/or
comparing a mid-infrared (MIR) spectrum of milk of the cow with a second reference MIR spectrum, wherein the second reference MIR spectrum is representative of a cow or cows having a poor likelihood of conception upon insemination; and
determining the likelihood of conception upon insemination of the cow on the basis of the comparison,
wherein the first reference MIR spectrum is derived from a cow or cows which have conceived at first insemination,
wherein the second reference MIR spectrum is derived from a cow or cows which did not conceive within a previous mating season and had only one insemination event, and
wherein the first reference MIR spectrum and/or the second reference MIR spectrum are not derived from a cow or cows which have conceived following two or more inseminations and which did not conceive but had more than one insemination event at last mating season.

2. The method of claim 1, wherein the cow will have a good likelihood of conception upon insemination if the MIR spectrum of the milk of the cow is more consistent with the first reference MIR spectrum than with the second reference MIR spectrum.

3. The method of claim 2, wherein the insemination is a second insemination.

4. The method of claim 1, wherein the cow will have a poor likelihood of conception upon insemination if the MIR spectrum of the milk of the cow is more consistent with the second reference MIR spectrum than with the first reference MIR spectrum.

5. The method of claim 4, wherein the insemination is a first insemination.

6. The method of claim 1, wherein the MIR spectra are compared using a statistical comparison.

7. The method of claim 6, wherein the statistical comparison is that of MIR spectral features of each MIR spectrum being compared.

8. The method of claim 7, wherein the MIR spectral features are individual wavenumbers of each MIR spectrum.

9. The method of claim 1, wherein the MIR spectrum of the milk of the cow is pre-treated prior to the comparison.

10. The method of claim 9, wherein the pre-treatment is removal of spectral regions 2998 to 3998 cm⁻¹, 1615 to 1652 cm⁻¹, and 649 to 925 cm⁻¹.

11. The method of claim 1, wherein the method further comprises:
comparing one or more further properties of the milk of the cow with a first reference for the one or more further properties of the milk, wherein the one or more further properties of the milk are related to fertility, and wherein the first reference for the one or more further properties of the milk is representative of a cow or cows having a good likelihood of conception upon insemination; and/or
comparing one or more further properties of the milk of the cow with a second reference for the one or more further properties of the milk, wherein the one or more further properties of the milk are related to fertility, and wherein the second reference for the one or more further properties of the milk is representative of a cow or cows having a poor likelihood of conception upon insemination; and
determining the likelihood of conception upon insemination of the cow on the basis of the comparison,
wherein the first reference for the one or more further properties of the milk is derived from a cow or cows which have conceived at first insemination,
wherein the second reference for the one or more further properties of the milk is derived from a cow or cows which did not conceive within a previous mating season and had only one insemination event, and
wherein the first reference and/or the second reference for the one or more further properties of the milk are not derived from a cow or cows which have conceived following two or more inseminations and which did not conceive but had more than one insemination event at last mating season.

12. The method of claim 11, wherein the one or more further properties of the milk comprise somatic cell count (SCC), fat content, protein content, lactose content, and fatty acid content.

13. The method of claim 1, wherein the method further comprises:
comparing one or more properties of the cow from which the milk was obtained with a first reference for the one or more properties of the cow, wherein the one or more properties of the cow are related to fertility, and wherein the first reference for the one or more properties of the cow is representative of a cow or cows having a good likelihood of conception upon insemination; and/or
comparing one or more properties of the cow from which the milk was obtained with a second reference for the one or more properties of the cow, wherein the one or more properties of the cow are related to fertility, and wherein the second reference for the one or more properties of the cow is representative of a cow or cows having a poor likelihood of conception upon insemination; and
determining the likelihood of conception upon insemination of the cow on the basis of the comparison,
wherein the first reference for the one or more properties of the cow is derived from a cow or cows which have conceived at first insemination,
wherein the second reference for the one or more properties of the cow is derived from a cow or cows which did not conceive within a previous mating season and had only one insemination event, and
wherein the first reference and/or the second reference for the one or more properties of the cow are not derived from a cow or cows which have conceived following two or more inseminations and which did not conceive but had more than one insemination event at last mating season.

14. The method of claim 13, wherein the one or more properties of the cow comprise:
(i) milk yield (MY) on the day of obtaining the milk of the cow;
(ii) previous lactation (305-day) milk yield;
(iii) previous lactation (305-day) fat yield;
(iv) previous lactation (305-day) protein yield;
(v) days in milk (DIM) of the cow on the day of obtaining the milk of the cow;
(vi) days from calving to insemination (DAI) of the cow;
(vii) calving age of the cow from a previous insemination;
(viii) fertility genomic estimated breeding value (GEBV); and
(ix) genotype of the cow.

15. The method of claim 1, wherein the milk of the cow is obtained from the cow before intended insemination.

16-24. (canceled)

25. The method of claim 1, further comprising:
selecting the cow for artificial insemination on the basis of the likelihood of conception.

26-42. (canceled)

43. The method of claim 1, further comprising classifying the fertility of the dairy cow,
wherein a cow having good fertility will have a good likelihood of conception upon insemination, and a cow having poor fertility will have a poor likelihood of conception upon insemination.

44-60. (canceled)

61. Software for use with a computer comprising a processor and memory for storing the software, the software comprising a series of coded instructions executable by the processor to carry out the method of claim 1.

62. (canceled)

63. A system for determining the likelihood of conception upon insemination of a dairy cow, for classifying the fertility of a dairy cow, or for selecting a dairy cow for artificial insemination, the system comprising:
a processor;
a memory; and
software resident in the memory accessible to the processor, the software comprising a series of coded instructions executable by the processor to carry out the method of claim 1.

64-70. (canceled)

71. A method of deriving a first reference and/or a second reference for a mid-infrared (MIR) spectrum of milk of a dairy cow, the method comprising:
dividing a cohort of dairy cows into three groups based on previous insemination outcomes, wherein the first group are cows which have conceived at first insemination, wherein the second group are cows which did not conceive within a previous mating season and had only one insemination event, and wherein the third group are cows which have conceived following two or more inseminations and which did not conceive but had more than one insemination event at last mating season;
obtaining or accessing a mid-infrared (MIR) spectrum of milk of each cow of the first group and/or the second group;
comparing the MIR spectrum of the milk of a cow in the first group with the MIR spectrum of the milk of each other cow in the first group to derive a first reference MIR spectrum; and/or
comparing the MIR spectrum of the milk of a cow in the second group with the MIR spectrum of the milk of each other cow in the second group to derive a second reference MIR spectrum,
wherein the first reference MIR spectrum is representative of cows having a good likelihood of conception or good fertility, and wherein the second reference MIR spectrum is representative of cows having a poor likelihood of conception or poor fertility.

72-82. (canceled)
